(19) United States
(12) Patent Application Publication
(10) Pub. No.: US 2026/0165810 A1
(43) Pub. Date: Jun. 18, 2026

(54) FORCE SENSING MEDICAL INSTRUMENT

(71) Applicant: Intuitive Surgical Operations, Inc., Sunnyvale, CA (US)

(72) Inventors: Lizmarie COMENENCIA ORTIZ, San Bruno, CA (US); David I. MOREIRA RIDSDALE, Saratoga, CA (US); Ashwinram SURESH, San Jose, CA (US); Kyle W. CLEMENTS, Pacifica, CA (US); Alan W. PETERSEN, Cupertino, CA (US); Cheng-Ling CHANG, Davis, CA (US)

(73) Assignee: Intuitive Surgical Operations, Inc., Sunnyvale, CA (US)

(21) Appl. No.: 19/129,124

(22) PCT Filed: Nov. 13, 2023
(86) PCT No.: PCT/US2023/079520

§ 371 Date: May 12, 2025

Related U.S. Application Data
(60) Provisional application No. 63/425,520, filed on Nov. 15, 2022.

(51) Int. Cl. A61B 90/00; A61B 18/00

(52) U.S. Cl. CPC A61B 90/06; A61B 18/00; A61B 2018/00178; A61B 2090/064

(57) ABSTRACT

Systems and methods are provided for control of a surgical system. A force sensing instrument for use with the surgical system includes an instrument shaft coupled to a proximal mechanical structure. A force sensor unit is coupled to a distal end portion of the instrument shaft. A circuit board is coupled to the proximal mechanical structure and configured to receive an output signal from the 2024/107653 force sensor unit. The force sensor unit is coupled to the circuit board via a sensor cable that is configured to mitigate electromagnetic interference with the output signal. Accordingly, the sensor cable has a middle portion and a set of electrical traces. The electrical traces include an electrical ground trace. An electrical shield surrounds the middle portion of the sensor cable and is communicatively coupled to the electrical ground trace.

### CROSS-REFERENCE TO RELATED APPLICATIONS

This application claims priority to and the filing date benefit of U.S. Provisional Ser. No. 63/425,520 , entitled “Force Sensing Medical Instrument,” filed Nov. 15, 2022, the disclosure of which is incorporated herein by reference in its entirety.

### BACKGROUND

The embodiments described herein relate to force sensing technology, and more specifically to force sensing technology adapted for use with teleoperated surgical systems. More particularly, the embodiments described herein relate to force sensing medical instruments for determining forces applied to the medical instrument in order to control a surgical system that includes a force feedback that may be provided to a system operator. Still more particularly, the embodiments described herein relate to the mitigation of electromagnetic interference when the force sensing medical instrument is exposed to an electrical field.

Known techniques for Minimally Invasive Surgery (MIS) employ instruments to manipulate tissue that can be either manually controlled or controlled via hand-held or mechanically grounded teleoperated medical systems that operate with at least partial computer-assistance (“telesurgical systems”). Many known MIS instruments include a therapeutic or diagnostic end effector (e.g., forceps, a cutting tool, or a cauterizing tool) mounted on an optional wrist mechanism at the distal end of a shaft. During an MIS procedure, the end effector, wrist mechanism, and the distal end of the shaft are typically inserted into a small incision or a natural orifice of a patient via a cannula to position the end effector at a work site within the patient's body. The optional wrist mechanism can be used to change the end effector's position and orientation with reference to the shaft to perform a desired procedure at the work site. In known instruments, motion of the instrument as a whole provides mechanical degrees of freedom (DOFs) for movement of the end effector and the wrist mechanisms generally provide the desired DOFs for movement of the end effector with reference to the shaft of the instrument. For example, for forceps or other grasping tools, known wrist mechanisms are able to change the pitch and yaw of the end effector with reference to the shaft. A wrist may optionally provide a roll DOF for the end effector, or the roll DOF may be implemented by rolling the shaft. An end effector may optionally have additional mechanical DOFs, such as grip or knife blade motion. In some instances, wrist and end effector mechanical DOFs may be combined. For example, U.S. Pat. No. 5,792,135 (filed May 16, 1997) discloses a mechanism in which wrist and end effector grip DOFs are combined.

Force sensing medical instruments are known and, together with associated telesurgical systems, may deliver haptic feedback during a MIS procedure to a surgeon performing the procedure. The haptic feedback may increase the surgeon's sense of immersion, realism, and intuitiveness while performing the procedure. For effective haptics rendering and accuracy, force sensors may be placed on a medical instrument and as close to the anatomical tissue interaction as possible. One approach is to include a force sensor unit having electrical sensor elements (e.g., strain gauges) at a distal end of a medical instrument shaft to measure strain imparted to the medical instrument. The measured strain can be used to determine the force imparted to the medical instrument and as input upon which the desired haptic feedback may be generated.

In some MIS procedures an electrical current is introduced to the surgical site, such as during electrosurgery. Electrosurgery refers broadly to a class of medical procedures that rely on the application of high frequency electrical energy, usually radio frequency energy, to patient tissue to achieve a number of possible effects, such as cutting, coagulation, necrosis, and the like. For example, in some MIS procedures tissue in the patient's body must be cauterized and severed. To perform such a procedure, end effector grips configured to apply bipolar or monopolar cauterizing energy are introduced to the surgical site to engage the target tissue, and electrical energy, such as radiofrequency energy, is delivered to the grips to cauterize the engaged tissue. Alternatively, in some instances surgeons have been known to engage tissue with electrically conductive end effector grips that are not specifically configured to apply electrical energy, and then place an actively charged electrode (such as an electrically charged end effector on a second instrument) in electrically conductive contact (i.e., direct electrical coupling) with the grips in order to apply electrosurgical energy to the tissue.

Force sensing instruments may be specifically designed to apply electrosurgical energy (e.g., a bipolar forceps instrument) or not designed to apply electrosurgical energy (e.g., a Cadiere forceps instrument). Regardless of whether a force sensing medical instrument is designed to apply electrosurgical energy, during certain MIS procedures the force sensing medical instrument can be exposed to an electrical field during an electrosurgical operation. And regardless of the approach used to apply electrosurgical energy to tissue-with an instrument specifically designed to apply electrosurgical energy or with an instrument not specifically designed to apply electrosurgical energy-electrical current associated with the electrosurgical energy can be conducted through or along various components of the force sensing medical instrument.

The exposure of the force sensing medical instrument to the electrical field can result in the generation of electromagnetic interference within the instrument that can affect signals from the force sensing instrument's force sensor unit. In turn, this effect on the signals can result in inaccurate indications of the forces acting on the force sensing medical instrument and the associated haptic feedback to the surgeon operating the force sensing instrument. Insofar as the haptic feedback is based on the indications of force on the instrument, it is desirable to mitigate the effects of the electromagnetic interference. Such mitigations are subject to the design and design constraints (e.g., component materials needed for strength or other mechanical properties, small component sizes required for surgery, etc.) of the force sensing instrument itself. For example, one approach has attempted to employ a Faraday cage around any components that could be affected. This required additional conductive components enclosing the entirety of the sensor along the complete instrument length, an effective grounding of the cage, and additional clearance. However, due to the additional components, this approach could adversely affect sensor performance (e.g., alignment, calibration, and/or robustness).

The magnitude and/or affect of the electromagnetic interference on the output of the force sensor unit can also depend, at least in part, on the positioning of various components of the force sensing medical instrument. For example, the conductive contact between the electrode and the force sensing medical instrument with an instrument not specifically designed to apply electrosurgical energy can result in the electrical current being conducted via a conductive component (e.g., a metal component such as a beam, a mechanical cable, and/or a shaft) of the force sensing medical instrument. The electrically conductive component can be separated from another electrically conductive component (e.g., strain sensors, strain gauges, and/or sensor cables) of the force sensing medical instrument by electrical insulation. However, the two electrically conductive components can become capacitively or inductively coupled (i.e., indirectly electrically coupled) when the current in the first component generates a current through the insulation into the second component. The magnitude of the generated current is affected, at least in part, by the positioning of the two conductive components and by the insulation therebetween. For example, a strain sensor can be mechanically coupled to an electrically conductive structure by an electrically insulative adhesive. In accordance with the principles of capacitive coupling, a current conducted by the structure can generate a current in the strain sensor through the electrically insulative adhesive. As another example, current can also be generated in the electrical traces of a sensor cable that carry the signal voltage from the strain sensor to a processor (e.g., one or more data processing components, a circuit board including one or more such components, a centralized or distributed data processing system including such components). The magnitude of the generated current can be affected by a distance between the sensor electronic components (e. g, the strain sensor or electrical traces) and other components, as well as the structure as determined by the thickness of the electrically insulative adhesive and other factors. Insofar as changes in the relatively low voltage of the strain sensor can be indicative of the forces acting on the force sensing medical instrument, the presence of electromagnetic interference (in the form of the generated current) in the output of the strain sensor can distort the force indications.

In addition to the capacitive coupling, electromagnetic interference can also result from the inductive coupling (e.g., antenna coupling or magnetic field coupling) of various components of the force sensing medical instrument. When inductively coupled, a magnetic field resulting from an electrical current in one conductor generates an electrical current in a second conductor. For example, a current can be generated via inductive coupling in a portion of the strain sensor and/or the sensor cable carrying signals from the strain sensor. The presence of the current generated by the inductive coupling is electromagnetic interference that can distort the indications of strain generated by the force sensor unit, resulting in discrepancies in the indications of the force acting on the force sensing medical instrument.

One electromagnetic interference problem may be caused by the long electrical connection between sensors at the distal end of the instrument and one or more other electrical components at the proximal end of the instrument needed to process or otherwise convey signals from the sensors to the user haptic feedback system. Individual electrical connection lines within an electrical connection cable between the instrument's distal and proximal ends may each act as antennas receiving unwanted electromagnetic energy. Although electromagnetic interference in the cable as a whole may be a problem, separate electrical connection lines within the cable may pick up differing amounts of electromagnetic interference, which can further degrade sensor signals for user haptic feedback.

Further, due to instrument physical design requirements, a cable with sufficient separate electrical connection lines between distal and proximal components must be small enough to fit within the small space constraints imposed by the small diameter of a minimally invasive surgical instrument's long shaft needed to minimize surgical incision size. On the other hand, the cable must be large enough to ensure the individual lines are large enough to be efficient electrical conductors for the sensor signals.

Still further, in addition to mitigating the effects of the electromagnetic interference within a minimally invasive surgical instrument's physical design constraints, it is desirable that the electronic components of a force sensing medical instrument be able to withstand post-surgical procedure processing (e.g., cleaning, sterilization by steam autoclaving, and the like) and remain resistant to fluid ingress after the post-surgical procedure processing. For example, some known electrical couplings between components (e.g., coupling of a conventional electrical conductor cable to a circuit board) are not able to withstand high temperatures or fluid flushing pressure that can occur during post-procedure processing. Additionally, in known electrical cables, an electrically insulative layer is often positioned between the layer containing the electrical couplings and the circuit board. The presence of this insulative layer can result in deformation of the layer containing the electrical couplings in order to bring the electrical couplings into contact with the circuit board. Such deformation of the layer can lead to a delamination, or pulling away, of a portion of the sensor cable, which then allows for the intrusion of fluids during post-procedure processing.

In view of the aforementioned, the art is continuously seeking new and improved systems and methods for control of a surgical system based on the accurate measurement of the strain imparted to the medical instrument.

### SUMMARY

This summary introduces certain aspects of the embodiments described herein to provide a basic understanding. This summary is not an extensive overview of the inventive subject matter, and it is not intended to identify key or critical elements or to delineate the scope of the inventive subject matter.

The systems and methods described herein facilitate the control of a surgical system when a force sensing medical instrument is exposed to an electrical field. In particular, the force sensing medical instrument uses a force sensor unit to measure forces affecting the force sensing medical instrument. The output of the force sensor unit is communicated to a circuit board (e.g., to a control board) and on to a controller of the system via a sensor cable. The sensor cable is configured to mitigate the effects of electromagnetic interference. With the electromagnetic effects being mitigated, the force sensor unit can transmit output strain signals from the force sensor unit that accurately indicate the forces affecting the force sensing medical instrument.

In one aspect, the present disclosure is directed to a force sensing medical instrument (“instrument”). The instrument can, for example, be used with a surgical system in the performance of a minimally invasive surgery. The instrument includes a proximal mechanical structure having a number of drive assemblies configured to cause a motion of an end effector of the instrument. For example, the instrument can include a set of capstans that are driven by a set of motors to alter the position of the end effector (e.g., a tool member) via a set of cables. An instrument shaft is coupled to the proximal mechanical structure. A force sensor unit is coupled to a distal end portion of the instrument shaft and configured to measure a force affecting the instrument. A circuit board is coupled to the proximal mechanical structure and configured to receive an output from the force sensor unit. A sensor cable that has a middle portion and a first set of electrical traces is communicatively coupled between the force sensor unit and the circuit board. In other words, the sensor cable facilitates communication between the force sensor unit and the circuit board. The first set of electrical traces includes an electrical ground trace, and the sensor cable includes an electrical shield that surrounds the middle portion of the sensor cable, with the electrical shield being communicatively coupled to the electrical ground trace.

In some embodiments, the sensor cable includes a proximal end portion, a distal end portion, a first layer, a proximal second layer, and a distal second layer. The first layer extends between the distal end portion of the sensor cable and the proximal end portion of the sensor cable. The first layer includes a proximal segment and a distal segment. The proximal second layer extends parallel to the proximal segment of the first layer. The distal second layer extends parallel to the distal segment of the first layer. The proximal segment of the first layer includes a proximal coupling interface. The proximal coupling interface includes a first set of electrically conductive contacts. The proximal segment of the first layer in the proximal end portion of the sensor cable is free of the first set of electrical traces. The proximal second layer is coupled to the proximal segment and contains a second set of electrical traces communicatively coupled to the set of conductive contacts.

In some embodiments, the middle portion of the sensor cable is free of the proximal second layer and the distal second layer. Additionally, the first set of electrical traces extends in a side-by-side planar configuration within the first layer through the middle portion of the sensor cable.

In some embodiments, the sensor cable includes a proximal transfer portion between the proximal end portion and the middle portion of the sensor cable. The proximal transfer portion includes a set of vias configured to communicatively couple the first set of electrical traces in the first layer in the middle portion to the second set of electrical traces in the proximal second layer.

In some embodiments, the first set of electrical traces includes a set of positive traces and a set of negative traces in the middle portion of the sensor cable. Each positive trace has a first cross-sectional area, and each negative trace has a second cross-sectional area. The first cross-sectional area is smaller than the second cross-sectional area.

In some embodiments, a first maximal resistance limit determines a minimal first cross-sectional area of the first cross-sectional area of the set of positive traces, and a second maximal resistance limit determines a minimal second cross-sectional area of the second cross-sectional area of the set of negative traces.

In some embodiments, a maximal sensor cable width defines a maximal combined cross-sectional area of each of the set of positive traces and the set of negative traces in the middle portion of the sensor cable. The maximal sensor cable width is defined at least in part by a passage clearance of the instrument shaft.

In some embodiments, the sensor cable includes a first electrically insulative layer, an electrically insulative base, and a second electrically insulative layer. The first electrically insulative layer is on a distal segment of the first layer and the middle portion of the sensor cable. The first electrically insulative layer is absent from the proximal segment of the first layer. The electrically insulative base extends between the distal end portion and the proximal end portion of the sensor cable. The second electrically insulative layer is on the distal second layer, the middle portion of the sensor cable, and the proximal second layer.

In some embodiments, the sensor cable includes a longitudinal axis extending between the proximal end portion and the distal end portion. The first set of conductive contacts is arranged along a contact axis that is parallel to the longitudinal axis of the sensor cable.

In some embodiments, the proximal coupling interface is an anisotropic conductive film coupling.

In some embodiments, the sensor cable includes a distal transfer portion between the middle portion and the distal end portion of the sensor cable. The distal transfer portion includes a set of vias configured to communicatively couple a first portion of the first set of electrical traces in the middle portion to a third set of electrical traces in the distal second layer.

In some embodiments, the distal segment of the first layer in the distal end portion of the sensor cable defines a distal coupling interface having a second set of conductive contacts coupled to the force sensor unit. A second portion of the first set of electrical traces are communicatively coupled to the distal coupling interface in the first layer. The third set of electrical traces in the distal second layer is coupled to the distal coupling interface. A linear arrangement of the second set of conductive contacts establishes an initial configuration of the third set of electrical traces and the second portion of the first set of electrical traces.

In some embodiments, the third set of electrical traces and the second portion of the first set of electrical traces are rearranged within the distal transfer portion to establish all electrical traces of the first set of electrical traces in a side-by-side, planar configuration within the first layer through the middle portion of the sensor cable.

In some embodiments, the middle portion of the sensor cable includes a first lateral side region and a second lateral side region separated by the electrical ground trace. The first set of electrical traces includes a set of positive traces and a set of negative traces in the middle portion of the sensor cable. The side-by-side, planar configuration includes the set of positive traces positioned within the first lateral side region and the set of negative traces positioned within the second lateral side region.

In some embodiments, the first set of electrical traces includes at least one positive trace and at least one negative trace in the middle portion of the sensor cable. The side-by-side, planar configuration includes the positive trace(s) arranged in a positive-negative pairing with the negative trace(s).

In some embodiments, the medical instrument includes a beam, and the beam includes a first face and a second face. The force sensor unit includes a strain sensor on the first face of the beam. The distal coupling interface is coupled to the strain sensor on the first face of the beam. The distal end portion of the sensor cable is coupled to the second face of the beam adjacent the first face of the beam. The distal coupling interface is formed with a pre-fold relative to a remainder of the distal end portion to align the distal coupling interface with the first face of the beam. A magnitude of the pre-fold corresponds to an angle between the first face of the beam and the second face of the beam.

In some embodiments, the sensor cable includes a balancing portion that extends distally from the distal end portion of the sensor cable. The balancing portion has a stiffness that corresponds to a stiffness of the distal end portion of the sensor cable. The balancing portion has an absence of electrical traces.

In some embodiments, the force sensor unit includes a strain sensor on a first face of a beam, and the strain sensor has a stiffness. The distal end portion of the sensor cable is coupled to a second face of the beam adjacent the first face. The sensor cable includes a stiffness-balancing tab that is coupled to a third face of the beam opposite the first face. The stiffness-balancing tab has a stiffness that corresponds to a stiffness of the strain sensor, and the stiffness-balancing tab has an absence of electrical traces.

In some embodiments, the strain sensor includes eight bridge circuits arranged as four bridge-circuit combinations. Each bridge circuit of the eight bridge circuits includes two strain gauges.

In some embodiments, the sensor cable has a first longitudinal length, and the first layer has a second longitudinal length. Also, the proximal second layer has a third longitudinal length, and the distal second layer has a fourth longitudinal length. The second longitudinal length equals the first longitudinal length. A combination of the third longitudinal length plus the fourth longitudinal length is less than the second longitudinal length of the first layer.

In some embodiments, the medical instrument includes an end effector and a wrist assembly. The end effector is coupled to the force sensor unit via the wrist assembly, and the medical instrument is configured to be operatively coupled to a surgical system.

### BRIEF DESCRIPTION OF THE DRAWINGS

FIG. 1 is a plan view of a minimally invasive teleoperated medical system according to an embodiment being used to perform a medical procedure such as surgery.

FIG. 2 is a perspective view of a user control console of the minimally invasive teleoperated surgery system shown in FIG. 1.

FIG. 3 is a perspective view of an optional auxiliary unit of the minimally invasive teleoperated surgery system shown in FIG. 1.

FIG. 4 is a front view of a manipulator unit, including a set of instruments, of the minimally invasive teleoperated surgery system shown in FIG. 1.

FIG. 5 is a perspective view of a force sensing medical instrument with a back cover removed for clarity according to an embodiment.

FIG. 6 is a side view of a portion of the instrument of FIG. 5 with an outer shaft removed.

FIG. 7 is a cross-sectional view of the instrument of FIG. 5 taken along line X-X in FIG. 5.

FIG. 8 is a schematic perspective view of a sensor cable for use with a force sensing instrument according to an embodiment.

FIG. 9 is a schematic side view of the sensor cable of FIG. 8.

FIG. 10A is a schematic cross-sectional view of a middle portion of the sensor cable of FIG. 8 taken along line X₁-X₁ in FIG. 9, depicting a first arrangement of electrical traces according to an embodiment.

FIG. 10B is a schematic cross-sectional view of a middle portion of the sensor cable of FIG. 8 taken along line X₁-X₁ in FIG. 9, depicting a second arrangement of electrical traces according to an embodiment.

FIG. 11 is a schematic cross-sectional view of a distal end portion of the sensor cable of FIG. 8 taken along line X₂-X₂ in FIG. 9.

FIG. 12 is a schematic cross-sectional view of a proximal end portion of the sensor cable of FIG. 8 taken along line X₃-X₃ in FIG. 9.

FIG. 13 is a perspective view of a sensor cable coupled between a force sensor unit and a circuit board according to an embodiment.

FIG. 14 is a perspective view of the sensor cable of FIG. 13 coupled to the force sensor unit.

FIG. 15A is a top view of a portion of the sensor cable of FIG. 14 with a first electrically insulative layer partially removed to reveal an electrical shield.

FIG. 15B is a top view of the portion of the sensor cable of FIG. 15A with the first electrically insulative layer and the electrical shield removed.

FIG. 16A is a top view of a portion of a first layer of the sensor cable of FIG. 15B.

FIG. 16B is a top view of a distal second layer and a distal coupling interface of the sensor cable of FIG. 15B.

FIG. 17 is a perspective view of the sensor cable of FIG. 13 coupled to the circuit board.

FIG. 18A is a top view of a portion of the sensor cable of FIG. 17 with a first electrically insulative layer partially removed to reveal an electrical shield.

FIG. 18B is a top view of the portion of the sensor cable of FIG. 18A with the first electrically insulative layer and the electrical shield removed.

FIG. 19A is a top view of a portion of a first layer of the sensor cable of FIG. 18B.

FIG. 19B is a top view of a distal second layer and a distal coupling interface of the sensor cable of FIG. 18B.

FIG. 20A is an electrical schematic illustration of one configuration of a strain sensor of the force sensor unit shown in FIG. 14.

FIG. 20B is an electrical schematic illustration of another example configuration of a strain sensor of the force sensor unit shown in FIG. 14.

FIG. 20C is a schematic illustration of an example layout of the strain gauges of the strain sensor of FIG. 20B.

FIG. 21 is a schematic illustration of a controller for use with a minimally invasive teleoperated surgery system according to an embodiment.

### DETAILED DESCRIPTION

Reference now will be made in detail to embodiments of the invention, one or more examples of which are illustrated in the drawings. Each example is provided by way of explanation of the invention, not limitation of the invention. In fact, it will be apparent to those skilled in the art that various modifications and variations can be made in the present invention without departing from the scope or spirit of the invention. For instance, features illustrated or described as part of one embodiment can be used with another embodiment to yield a still further embodiment. Thus, it is intended that the present invention covers such modifications and variations as come within the scope of the appended claims and their equivalents.

The embodiments described herein can advantageously be used in a wide variety of grasping, cutting, and manipulating operations associated with minimally invasive surgery. The medical instruments or devices of the present application enable motion in three or more degrees of freedom (DOFs). For example, in some embodiments, an end effector of the medical instrument can move with reference to the main body of the instrument in three mechanical DOFs, e.g., pitch, yaw, and roll (shaft roll). There may also be one or more mechanical DOFs in the end effector itself, e.g., two jaws, each rotating with reference to a clevis (2 DOFs) and a distal clevis that may rotate with reference to a proximal clevis (one DOF). Thus, in some embodiments, the medical instruments or devices of the present application may enable motion in six DOFs. The embodiments described herein further may be used to deliver haptic feedback to a system operator based on a load indication from the force sensor unit.

Generally, the present disclosure is directed to systems and methods for controlling a surgical system (system), such as a teleoperated minimally invasive surgery system. In particular, the present disclosure includes a force sensor unit that is communicatively coupled to a circuit board via a sensor signal cable. The sensor cable is configured to mitigate electromagnetic interference. The sensor cable can be employed with a force sensing medical instrument (instrument) to communicate indications of a force affecting the instrument. This indication of the force(s) can be used by the system to deliver haptic feedback to a user control unit of the system.

As described herein, the force sensor unit includes a strain sensor coupled to a resiliently deformable beam. The beam is configured to deform in response to a load affecting a distal end portion of the instrument. The strain sensor includes strain gauges that measure the resultant strain in the beam due to the deflection. The strain sensor indicates the strain magnitude in the form of relatively small voltage differentials. In some embodiments, the strain gauges are arranged in a split-bridge configuration (e.g., a split Wheatstone bride) with one half of the split-bridge being coupled to a positive trace configured to carry a signal at a positive electrical potential and the other half being coupled to a negative trace configured to carry a signal at a negative electrical potential. The voltage differential, as opposed to an absolute voltage, between the signal carried by the positive trace and the signal carried by the negative trace is indicative of the measured strain magnitude in the absence of electromagnetic interference.

During certain procedures, portions of the instrument, such as the force sensor unit, can be exposed to an electrical field. This exposure can result in the development of electromagnetic interference that can affect the signals in the positive and/or negative traces. For example, a current conducted through a portion of the instrument, such as the instrument shaft, can induce an unintended current in another portion of the instrument, such as the sensor cable. The induced current can result from capacitive coupling and/or inductive coupling between the various conductive components of the instrument. The magnitude of the induced current, and thus the magnitude of the electromagnetic interference, can be affected by the positions and/or orientations of the various conductive components of the instrument relative to one another, as well as the presence of grounded shielding. When the magnitude of the electromagnetic interference (i.e., the induced current(s)) in one of the traces is greater than the magnitude of the electromagnetic interference in other trace, then the voltage differential, and thus the measured strain magnitude, is distorted. However, when a difference between the magnitude of the electromagnetic interference in each of the traces is minimized, the effect of electromagnetic interference in one trace is substantially cancelled out by the electromagnetic interference in the other trace, and vice versa. Accordingly, it is desirable to mitigate the effects of the electromagnetic interference by minimizing a differential between the induced current in the positive trace coupled to one half of the split-bridge and the induced current in the corresponding negative trace coupled to the other half of the split-bridge.

Insofar as changes in the relatively low voltage of the strain sensor can be indicative of the forces acting on the instrument, it is desireable to minimize the resistance of the sensor cable. To this end, maximal resistance limits can establish minimum acceptable cross-sectional areas for the positive and negative traces of the sensor cable. However, while increasing the cross-sectional areas of a trace lowers the resistance of the trace (for a given trace material), the maximal dimensions of the sensor cable, and thus the maximal combined cross-sectional areas of the traces, are limited by the internal structure of the instrument shaft through which the cable is routed. For example, the sensor cable can have a maximal width that is defined at least in part by a passage clearance of the instrument shaft. Additionally, it is desirable that the sensor cable be sufficiently flexible to facilitate the movements of the end effector during an operation. To that end, the electrical traces can be arranged in a single side-by-side planar configuration in a portion of the sensor cable within the instrument shaft. To mitigate the effects of electromagnetic interference, this portion of the sensor cable can be electrically shielded via a shield layer that is coupled to a ground trace. Further mitigation of the effects electromagnetic interference can be achieved via various arrangements of the positive and negative traces within the sensor cable.

As described herein, it is also desirable that the sensor cable be formed to facilitate post-procedure processing (e.g., autoclaving) of the medical instrument. To that end, the sensor cable can be formed to facilitate sealed couplings (e.g., connections or contacts) with the circuit board and the force sensor unit. For example, the sensor cable can include a layer that has a set of electrical couplings that are positioned to be coupled to the circuit board. Specifically, the sensor cable described herein can be formed so that the sensor cable can be coupled to the circuit board without flexing or deformation of the layer. This arrangement minimizes residual strain in the sensor cable near the point of the electrical couplings, thereby reducing the likelihood that the sensor cable will pull away from the circuit board.

Additionally, as described herein, a portion of the sensor cable can be coupled to the beam of the force sensor unit. However, this coupling can increase the stiffness of the corresponding portion of the beam. This increase in stiffness can affect the degree of deflection of the corresponding portion of the beam, resulting in a distortion of the sensed strain at various points along the beam. Therefore, the sensor cable can include a balancing portion that extends distally from the distal end portion of the sensor cable. This balancing portion can have a stiffness that corresponds to the stiffness of the portion of the sensor cable coupled to the beam but does not include any electrical traces. For example, the balancing portion can have a stiffness that is equal to (or substantially equal to) the stiffness of the portion of the sensor cable coupled to the beam. As another example, the balancing portion can have a stiffness that, along with the portion of the beam to which the balancing portion is coupled to, produces similar deflection characteristics to that produced by the portion of the sensor cable coupled to the beam. Accordingly, the increase in stiffness of the beam can be uniform along the length of the beam, resulting in uniformity of the sensed strain along the beam. In other words, the balancing portion can be employed to reduce the effects of any stiffness concentrations that result from the positioning of the sensor cable on the beam.

As used herein, the term “about” when used in connection with a referenced numeric indication means the referenced numeric indication plus or minus up to 10 percent of that referenced numeric indication. For example, the language “about 50″ covers the range of 45 to 55. Similarly, the language ”about 5″ covers the range of 4.5 to 5.5.

As used in this specification and the appended claims, the word “distal” refers to direction towards a work site, and the word “proximal” refers to a direction away from the work site. Thus, for example, the end of a tool that is closest to the target tissue would be the distal end of the tool, and the end opposite the distal end (i.e., the end manipulated by the user or coupled to the actuation shaft) would be the proximal end of the tool.

Further, specific words chosen to describe one or more embodiments and optional elements or features are not intended to limit the invention. For example, spatially relative terms-such as “beneath”, “below”, “lower”, “above”, “upper”, “proximal”, “distal”, and the like-may be used to describe the relationship of one element or feature to another element or feature as illustrated in the figures. These spatially relative terms are intended to encompass different positions (i.e., translational placements) and orientations (i.e., rotational placements) of a device in use or operation in addition to the position and orientation shown in the figures. For example, if a device in the figures is turned over, elements described as “below” or “beneath” other elements or features would then be “above” or “over” the other elements or features. Thus, the term “below” can encompass both positions and orientations of above and below. A device may be otherwise oriented (e.g., rotated 90 degrees or at other orientations) and the spatially relative descriptors used herein interpreted accordingly. Likewise, descriptions of movement along (translation) and around (rotation) various axes include various spatial device positions and orientations. The combination of a body's position and orientation define the body's pose (e.g., a kinematic pose).

Similarly, geometric terms, such as “parallel”, “perpendicular”, “round”, or “square”, are not intended to require absolute mathematical precision, unless the context indicates otherwise. Instead, such geometric terms allow for variations due to manufacturing or equivalent functions. For example, if an element is described as “round” or “generally round,” a component that is not precisely circular (e.g., one that is slightly oblong or is a many-sided polygon) is still encompassed by this description.

In addition, the singular forms “a”, “an”, and “the” are intended to include the plural forms as well, unless the context indicates otherwise. The terms “comprises”, “includes”, “has”, and the like specify the presence of stated features, steps, operations, elements, components, etc. but do not preclude the presence or addition of one or more other features, steps, operations, elements, components, or groups.

Unless indicated otherwise, the terms “apparatus,” “medical device,” “instrument,” “medical instrument,” “surgical instrument,” and variants thereof, can be interchangeably used.

Inventive aspects are described with reference to a teleoperated surgical system. An example architecture of such a teleoperated surgical system is the da Vinci® surgical system commercialized by Intuitive Surgical, Inc., Sunnyvale, California. Knowledgeable persons will understand, however, that inventive aspects disclosed herein may be embodied and implemented in various ways, including computer-assisted, non-computer-assisted, and hybrid combinations of manual and computer-assisted embodiments and implementations. Implementations are merely presented as examples, and they are not to be considered as limiting the scope of the inventive aspects disclosed herein. As applicable, inventive aspects may be embodied and implemented in both relatively smaller, hand-held, hand-operated devices and relatively larger systems that have additional mechanical support.

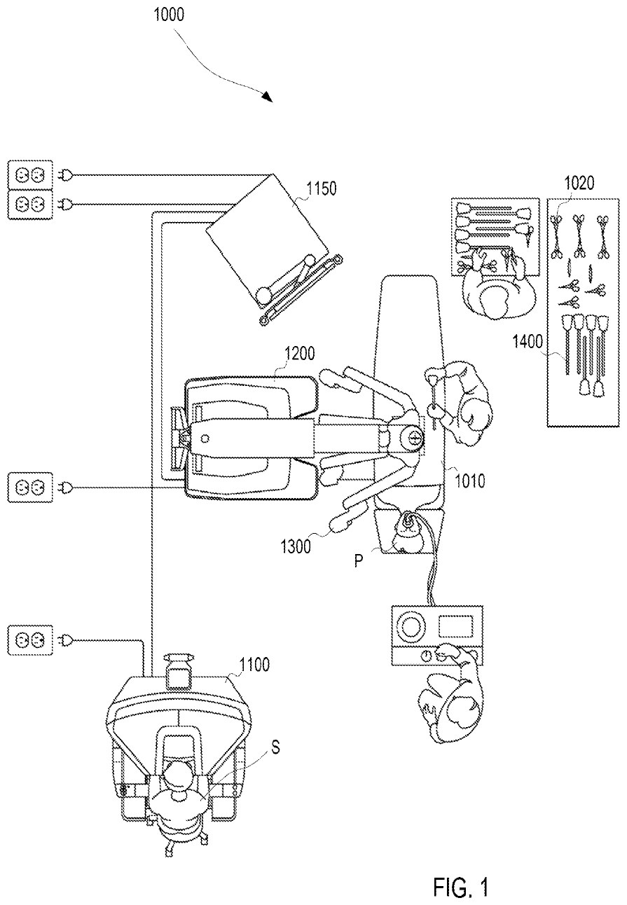

FIG. 1 is a plan view illustration of a teleoperated surgical system (“system”) 1000 that operates with at least partial computer assistance (a “telesurgical system”). Both telesurgical system 1000 and its components are considered medical devices. Telesurgical system 1000 is a Minimally Invasive Robotic Surgical (MIRS) system used for performing a minimally invasive diagnostic or surgical procedure on a Patient P who is lying on an Operating table 1010. The system can have any number of components, such as a user control unit 1100 for use by an operator of the system, such as a surgeon or other skilled clinician S, during the procedure. The MIRS system 1000 can further include a manipulator unit 1200 (popularly referred to as a surgical robot) and an optional auxiliary equipment unit 1150. The manipulator unit 1200 can include an arm assembly 1300 and a surgical instrument tool assembly removably coupled to the arm assembly. The manipulator unit 1200 can manipulate at least one removably coupled medical instrument (instrument) 1400 (e.g., a force sensing medical instrument) through a minimally invasive incision in the body or natural orifice of the patient P while the surgeon S views the surgical site and controls movement of the instrument 1400 through control unit 1100. An image of the surgical site is obtained by an endoscope (not shown), such as a stereoscopic endoscope, which can be manipulated by the manipulator unit 1200 to orient the endoscope. The auxiliary equipment unit 1150 can be used to process the images of the surgical site for subsequent display to the Surgeon S through the user control unit 1100. The number of instruments 1400 used at one time will generally depend on the diagnostic or surgical procedure and the space constraints within the operating room, among other factors. If it is necessary to change one or more of the instruments 1400 being used during a procedure, an assistant removes the instrument 1400 from the manipulator unit 1200 and replaces it with another instrument 1400 from a tray 1020 in the operating room. Although shown as being used with the instruments 1400, any of the instruments described herein can be used with the system 1000.

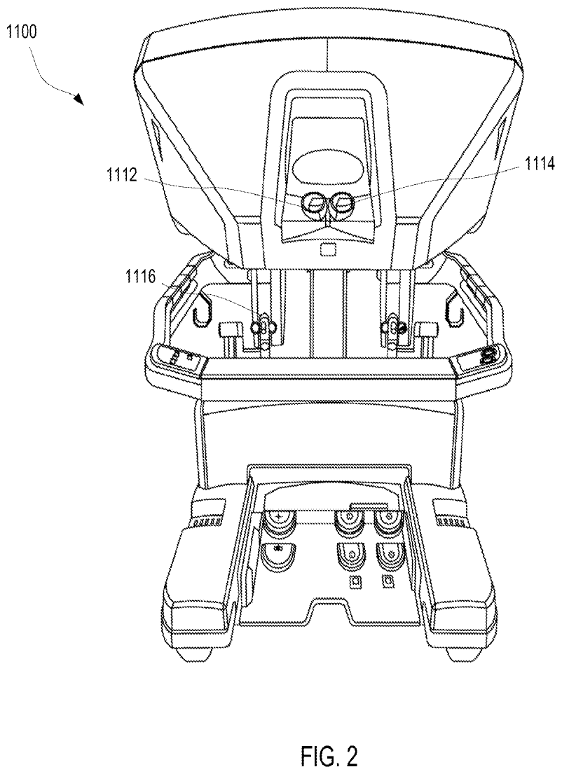

FIG. 2 is a perspective view of the user control unit 1100. The user control unit 1100 includes a left eye display 1112 and a right eye display 1114 for presenting the surgeon S with a coordinated stereoscopic view of the surgical site that enables depth perception. The user control unit 1100 further includes one or more input control devices 1116 (input device), which in turn cause the manipulator unit 1200 (shown in FIG. 1) to manipulate one or more tools. The input devices 1116 provide at least the same degrees of freedom as instruments 1400 with which they are associated to provide the surgeon S with telepresence, or the perception that the input devices 1116 are integral with (or are directly connected to) the instruments 1400. In this manner, the user control unit 1100 provides the surgeon S with a strong sense of directly controlling the instruments 1400. To this end, position, force, strain, or tactile feedback sensors (not shown) or any combination of such sensations, from the instruments 1400 back to the surgeon's hand or hands through the one or more input devices 1116.

The user control unit 1100 is shown in FIG. 1 as being in the same room as the patient so that the surgeon S can directly monitor the procedure, be physically present if necessary, and speak to an assistant directly rather than over the telephone or other communication medium. In other embodiments, however, the user control unit 1100 and the surgeon S can be in a different room, a completely different building, or other location remote from the patient, allowing for remote surgical procedures.

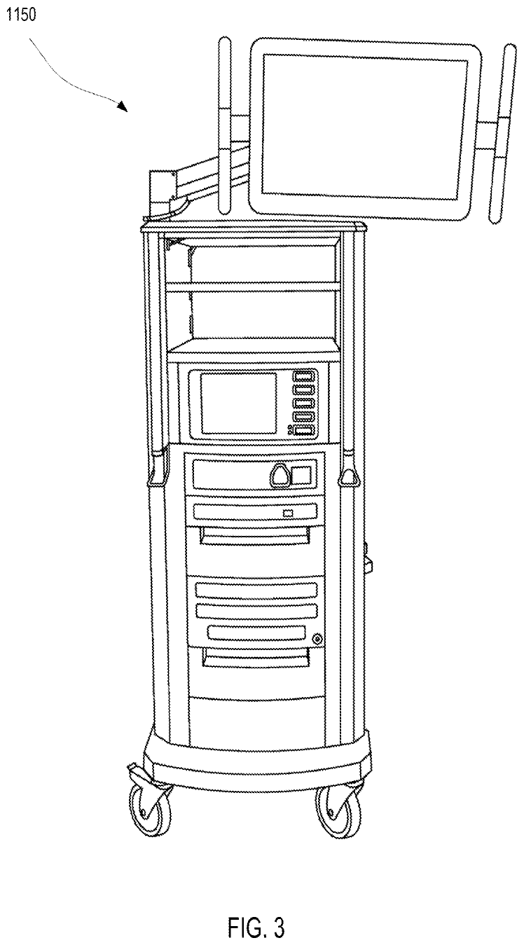

FIG. 3 is a perspective view of the auxiliary equipment unit 1150. The auxiliary equipment unit 1150 can be coupled with the endoscope (not shown) and can include one or more processors to process captured images for subsequent display, such as via the user control unit 1100, or on another suitable display located locally (e.g., on the unit 1150 itself as shown, on a wall-mounted display) and/or remotely. For example, where a stereoscopic endoscope is used, the auxiliary equipment unit 1150 can process the captured images to present the surgeon S with coordinated stereo images of the surgical site via the left eye display 1112 and the right eye display 1114. Such coordination can include alignment between the opposing images and can include adjusting the stereo working distance of the stereoscopic endoscope. As another example, image processing can include the use of previously determined camera calibration parameters to compensate for imaging errors of the image capture device, such as optical aberrations.

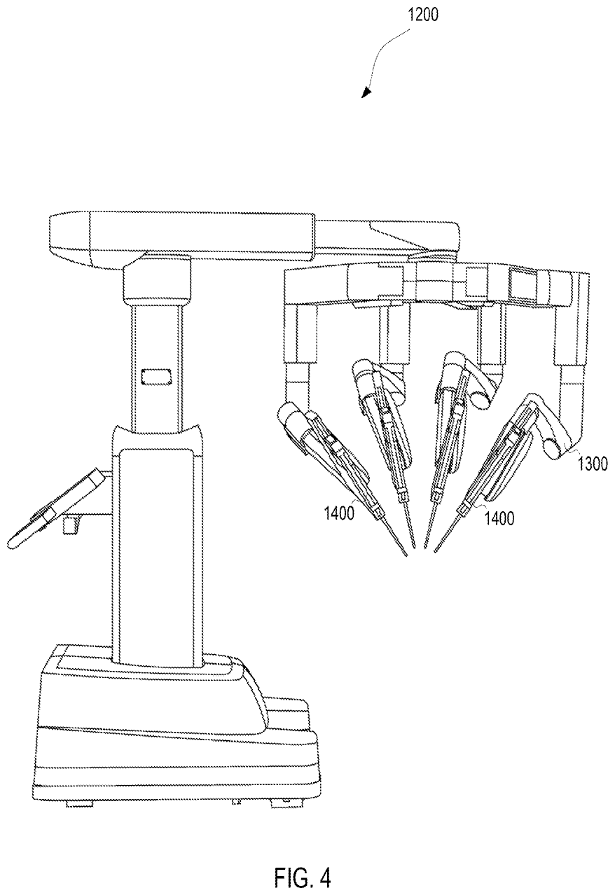

FIG. 4 shows a front perspective view of the manipulator unit 1200. The manipulator unit 1200 includes the components (e.g., arms, linkages, motors, sensors, and the like) to provide for the manipulation of the instruments 1400 and an imaging device (not shown), such as a stereoscopic endoscope, used for the capture of images of the site of the procedure. Specifically, the instruments 1400 and the imaging device can be manipulated by teleoperated mechanisms having one or more mechanical joints. Moreover, the instruments 1400 and the imaging device are positioned and manipulated through incisions or natural orifices in the patient P in a manner such that a center of motion remote from the manipulator and typically located at a position along the instrument shaft is maintained at the incision or orifice by either kinematic mechanical or software constraints. In this manner, the incision size can be minimized.

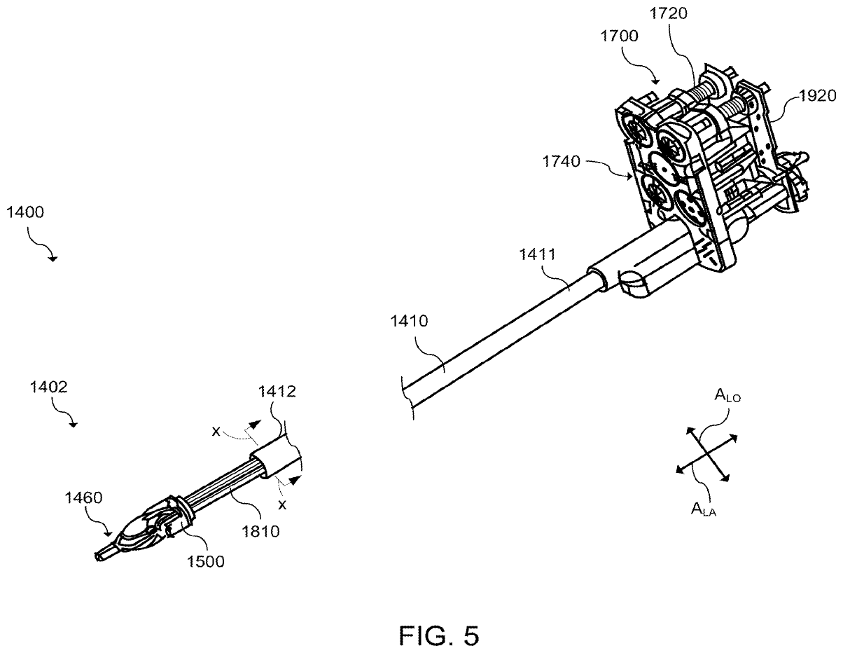

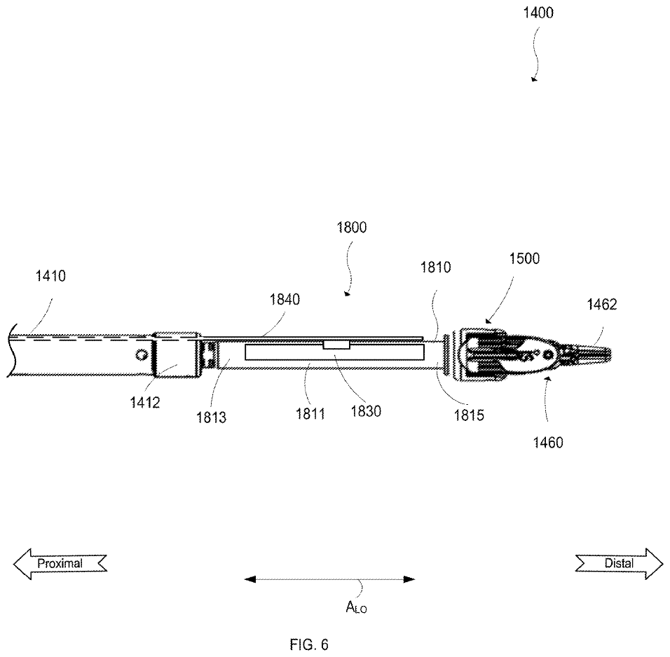

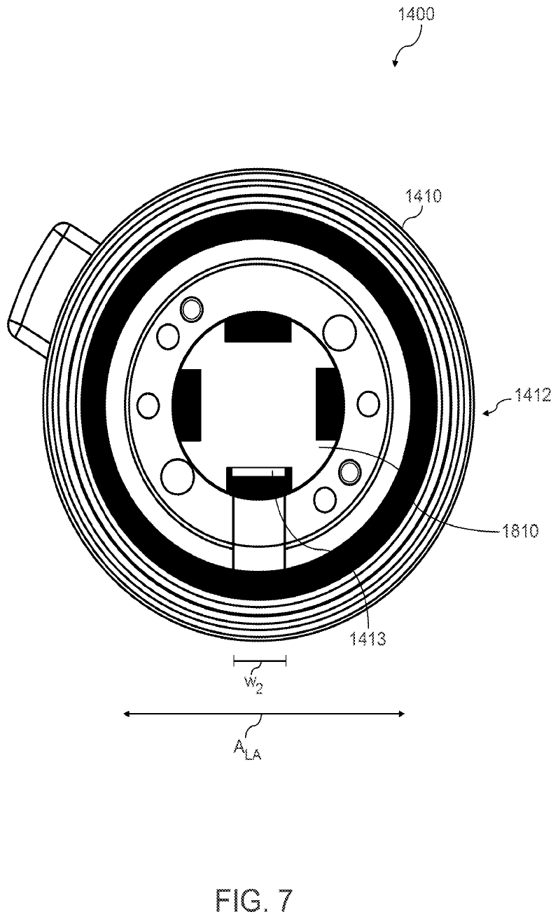

Referring now to FIGS. 5-7, a perspective view of the instrument 1400 is depicted in FIG. 5, a side view of a portion of the instrument 1400 with an outer shaft portion removed is depicted in FIG. 6, and a cross-sectional view of the instrument 1400 is depicted in FIG. 7. In some embodiments, the instrument 1400 or any of the components therein are optionally parts of a surgical system that performs surgical procedures, and which can include a manipulator unit, a series of kinematic linkages, a set of cannulas, or the like. The instrument 1400 (and any of the instruments described herein) can be used in any suitable surgical system, such as the MIRS system 1000 shown and described above. As shown in FIG. 5, the instrument 1400 includes a proximal mechanical structure 1700 (depicted with an outer cover removed), a shaft 1410, a distal end portion 1402, and a set of cables (not shown). The cables function as tension elements that couple the proximal mechanical structure 1700 to the distal end portion 1402. In some embodiments, the distal end portion 1402 includes a distal wrist assembly 1500 and a distal end effector 1460. The instrument 1400 is configured such that movement of one or more of the cables produces movement of the end effector 1460 (e.g., pitch, yaw, or grip) about axes of a beam coordinate system.

The proximal mechanical structure 1700 is configured to be removably coupled to the arm assembly 1300 manipulator unit 1200 (FIG. 4). The manipulator unit 1200 includes teleoperated actuators (e.g., motors with coupled drive discs) to provide controller motions to the instrument 1400, which translates into a variety of movements of a tool or tools at a distal end portion 1402 of the instrument 1400. When an instrument 1400 is coupled to the arm assembly 1300, input provided by a surgeon S to the user control unit 1100 (a “master” command) is translated into a corresponding action by the instrument 1400 (a “slave” response) via drive discs of the arm assembly 1300 that are operatively coupled instrument discs 1740 on the instrument 1400.

In some embodiments, the proximal mechanical structure 1700 includes a circuit board 1920 (e.g., a control board). The circuit board 1920 is communicatively coupled to a force sensor unit 1800 via a sensor cable 1840. The circuit board 1920 is configured to provide a voltage input to the strain sensor 1830 of the force sensor unit 1800 and to receive an output signal from the strain sensor 1830 that is indicative of a force affecting the distal end portion 1402 of the instrument 1400. Further details regarding the circuit board 1920 are provided in U.S. Provisional Ser. No. 63/425,524 , filed Nov. 15, 2022, the disclosure of which is incorporated herein by reference for all purposes. Further details regarding the force sensor unit 1800 are provided in co-pending U.S. Provisional Ser. No. 63/425,518 , filed Nov. 15, 2022, the disclosure of which is incorporated herein by reference for all purposes.

Moreover, although the proximal mechanical structure 1700 is shown as including capstans 1720, in other embodiments, a mechanical structure can include one or more linear actuators that produce translation (linear motion) of a portion of the cables. Such proximal mechanical structures can include, for example, a gimbal, a lever, or any other suitable mechanism to directly pull (or release) an end portion of any of the cables. For example, in some embodiments, the proximal mechanical structure 1700 can include any of the proximal mechanical structures or components described in U.S. Patent Application Pub. No. US 2015/0047454 A1 (filed Aug. 15, 2014), entitled “Lever Actuated Gimbal Plate,” or U.S. Pat. No. 6,817,974 B2 (filed Jun. 28, 2001), entitled “Surgical Tool Having Positively Positionable Tendon-Actuated Multi-Disc Wrist Joint,” each of which is incorporated herein by reference in its entirety.

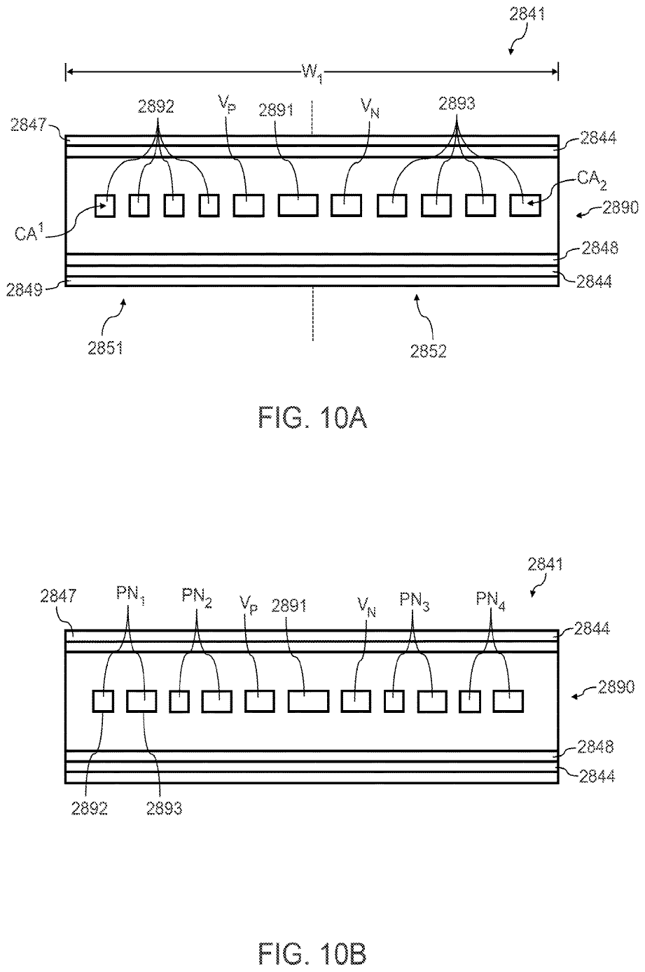

Referring still to FIGS. 5-7, the shaft 1410 can be any suitable elongated shaft that is coupled to the wrist assembly 1500 and to the proximal mechanical structure 1700. Specifically, the shaft 1410 includes a proximal end 1411 that is coupled to the proximal mechanical structure 1700, and a distal end portion 1412 that is coupled to the wrist assembly 1500 (e.g., a proximal link of the wrist assembly 1500). The shaft 1410 defines a passageway or series of passageways through which the cables and other components can be routed from the proximal mechanical structure 1700 to the wrist assembly 1500. For example, as depicted in FIG. 7, the shaft 1410 defines a sensor-cable passageway 1413 through which the sensor cable 1840 is routed. The sensor-cable passageway 1413 has a passage clearance W₂ that can define a maximal sensor cable width (e.g., the maximal sensor cable width W₁ as depicted in FIG. 10A).

In some embodiments, the shaft 1410 can be formed, at least in part with, for example, an electrically conductive material such as stainless steel. In such embodiments, the shaft may include any of an inner insulative cover or an outer insulative cover. Thus, the shaft 1410 can be a shaft assembly that includes multiple different components. For example, the shaft 1410 can include (or be coupled to) a spacer that provides the desired fluid seals, electrical isolation features, and any other desired components for coupling the wrist assembly 1500 to the shaft 1410. Similarly stated, although the wrist assembly 1500 (and other wrist assemblies or links described herein) are described as being coupled to the shaft 1410, it is understood that any of the wrist assemblies or links described herein can be coupled to the shaft via any suitable intermediate structure, such as a spacer and a cable guide, or the like.

As depicted in FIG. 6, the instrument 1400 (e.g., the force sensing medical instrument) includes a force sensor unit 1800. The force sensor unit includes a beam 1810, with one or more strain sensors 1830. The strain sensor 1830 can include a set of strain gauges (e.g., tension strain gauge resistor(s) or compression strain gauge resistor(s)) arranged as at least one bridge circuit (e.g., Wheatstone bridges) mounted on a surface along the beam 1810. In some embodiments, the end effector 1460 can be coupled at a distal end portion 1815 of the beam 1810 (e.g., at a distal end portion 1402 of the surgical instrument 1400) via the wrist assembly 1500. The shaft 1410 includes a distal end portion 1412 (e.g., an inner shaft) that is coupled to a proximal end portion 1813 of the beam 1810. In some embodiments, the distal end portion 1412 of the shaft 1410 is coupled to the proximal end portion 1813 of the beam 1810 via another coupling component (such as an anchor or coupler, not shown). In some embodiments, the force sensor unit 1800 can include any of the structures or components described in U.S. Patent Application Pub. No. US 2020/0278265 A1 (filed May. 13, 2020), entitled “Split Bridge Circuit Force Sensor,” which is incorporated herein by reference in its entirety.

In some embodiments, the end effector 1460 can include at least one tool member 1462 having a contact portion configured to engage or manipulate a target tissue during a surgical procedure. For example, in some embodiments, the contact portion can include an engagement surface that functions as a gripper, cutter, tissue manipulator, or the like. In other embodiments, the contact portion can be an energized tool member that is used for cauterization or electrosurgical procedures. The end effector 1460 may be operatively coupled to the proximal mechanical structure 1700 such that the tool member 1462 rotates relative to shaft 1410. In this manner, the contact portion of the tool member 1462 can be actuated to engage or manipulate a target tissue during a surgical procedure. The tool member 1462 (or any of the tool members described herein) can be any suitable medical tool member. Moreover, although only one tool member 1462 is identified, as shown, the instrument 1400 can include two tool members that cooperatively perform gripping or shearing functions. In other embodiments, an end effector can include more than two tool members.

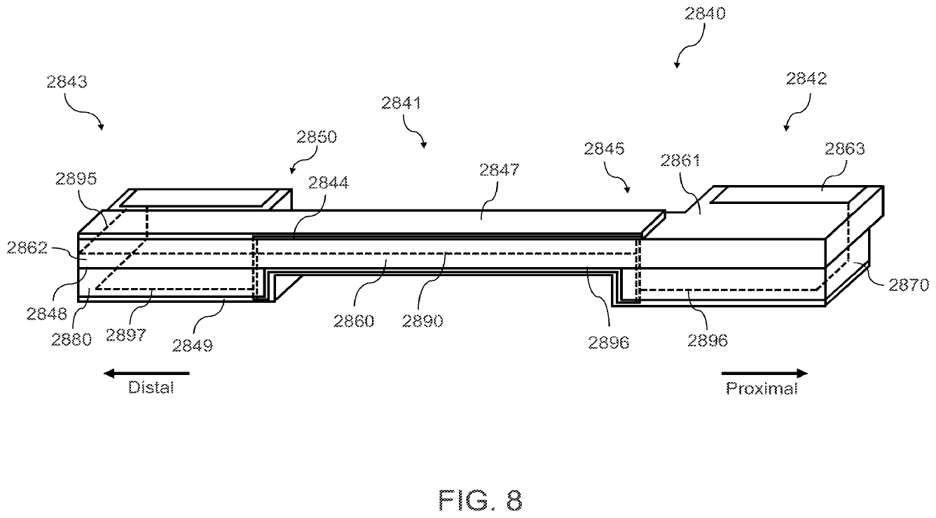

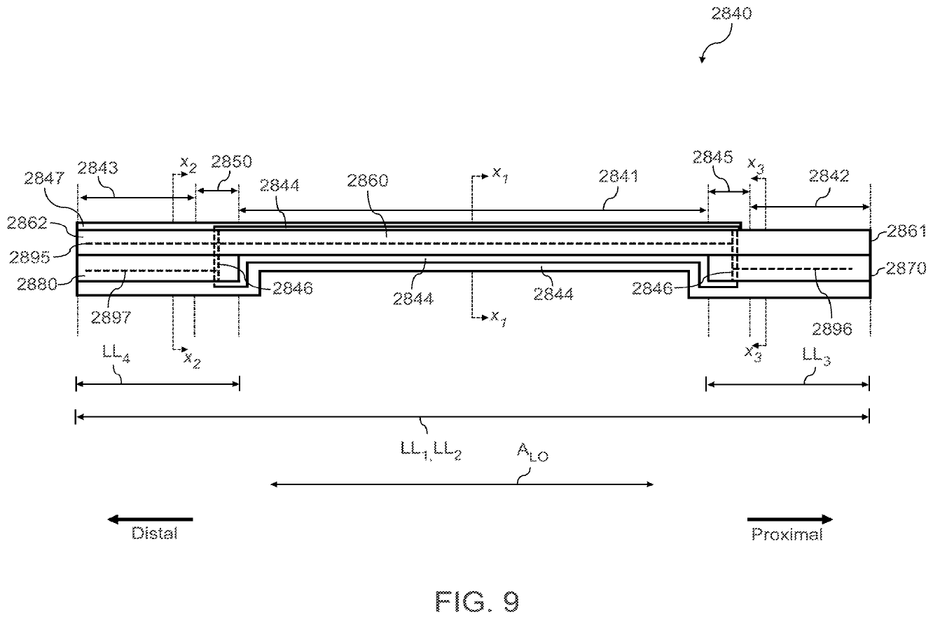

FIG. 8 is a schematic perspective view and FIG. 9 is a schematic side view of a sensor cable 2840 for use with the instrument 1400 (or any of the instruments described herein) according to various embodiments. The sensor cable 2840 can, for example, be used to communicatively couple the force sensor unit 1800 (or any of the force sensor units described herein) to the circuit board 1920 (or any of the circuit boards described herein). Therefore, the sensor cable 2840 can, in some embodiments, extend within the instrument shaft 1410 between the proximal mechanical structure 1700 and the force sensor unit 1800. It should be appreciated that some embodiments of the sensor cable 2840 do not require each and every optional element, component, and/or feature depicted.

As depicted, the sensor cable 2840 includes a middle portion 2841 that is between a proximal end portion 2842 and a distal end portion 2843. The middle portion 2841 of the sensor cable 2840 includes a first set of electrical traces 2890. The first set of electrical traces 2890 can be communicatively coupled between a force sensor unit (not shown) that is configured to measure a force affecting the instrument and the circuit board (not shown) that is configured to receive an output from the force sensor unit. The first set of electrical traces 2890 includes an electrical ground trace 2891 (FIG. 10A). The electrical ground trace 2891 can electrically ground the force sensor unit to the circuit board. In some embodiments, the sensor cable 2840 includes an electrical shield 2844 that surrounds the middle portion 2841 of the sensor cable 2840.

In some embodiments, the electrical shield 2844 is communicatively coupled to the electrical ground trace 2891. The electrical shield 2844 can, for example, be an electrically conductive material (e.g., a metallic film, spiraled wire strands, or other similar conductive structures) that is radially outward from the first set of electrical traces 2890. In some embodiments, the electrical shield 2844 can, as depicted, extend along an upper lateral face and a lower lateral face of the middle portion 2841. However, in additional embodiments, the electrical shield 2844 can surround the middle portion 2841. The electrical shield 2844 can, for example, limit the transmission of electromagnetic radiation to the first set of electrical traces 2890 and, therefore, mitigate the impact of electromagnetic interference on the signals transmitted by the sensor cable 2840. In other words, the electrical shield 2844 can electrically isolate the first set of electrical traces 2890 from other conductive components of the instrument, such as the instrument shaft.

Referring still to FIGS. 8 and 9, in some embodiments the sensor cable 2840 includes a first layer 2860, a proximal second layer 2870, and a distal second layer 2880. The first layer 2860 extends between the distal end portion 2843 of the sensor cable 2840 and the proximal end portion 2842 of the sensor cable 2840. The first layer 2860 includes a proximal segment 2861 that is within the proximal end portion 2842 of the sensor cable 2840. The first layer 2860 also includes a distal segment 2862 that is within the distal end portion 2843 of the sensor cable 2840. In other words, the first layer 2860 extends along the entirety of the sensor cable 2840. In some embodiments, the proximal second layer 2870 extends parallel to the proximal segment 2861 of the first layer 2860 within the proximal end portion 2842 of the sensor cable 2840. Similarly, the distal second layer 2880 extends parallel to the distal segment 2862 of the first layer 2860 within the distal end portion 2843 of the sensor cable 2840. In some embodiments, the middle portion 2841 of the sensor cable 2840 is free of the proximal second layer 2870 and the distal second layer 2880. In such an embodiment, the first set of electrical traces 2890 can extend in a side-by-side planar configuration within the first layer 2860 through the middle portion 2841 of the sensor cable 2840. Said another way, neither the proximal second layer 2870 nor the distal second layer 2880 extend the entirety of the sensor cable 2840.

In some embodiments, the sensor cable 2840 has a first longitudinal length LL₁. The first longitudinal length LL₁ corresponds to the entire longitudinal length of the sensor cable 2840. The first layer 2860 has a second longitudinal length LL₂. The second longitudinal length LL₂ equals the first longitudinal length LL₁. In other words, since the first layer 2860 extends the length of the sensor cable 2840, the first layer 2860 and the sensor cable 2840 have the same longitudinal length. The proximal second layer 2870 has a third longitudinal length LL₃, and the distal second layer 2880 as a fourth longitudinal length LL₄. A combination of the third longitudinal length LL₃ and the fourth longitudinal length LL₄ is less than the second longitudinal length LL₂ of the first layer 2860. In other words, the combined longitudinal length of the proximal second layer 2870 and a distal second layer 2880 is less than the longitudinal length of the first layer 2860. It should be appreciated that the longitudinal length of the sensor cable 2840 corresponds to a length along the longitudinal axis A_{LO}.

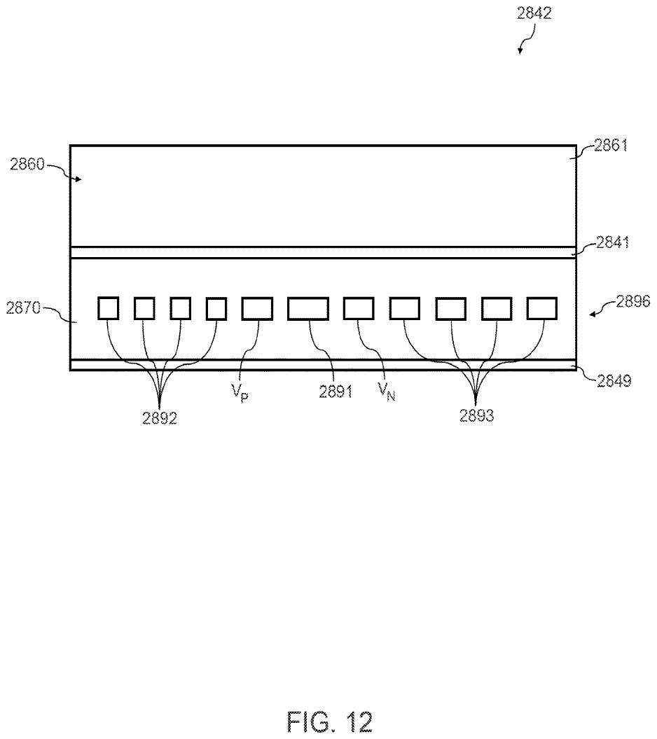

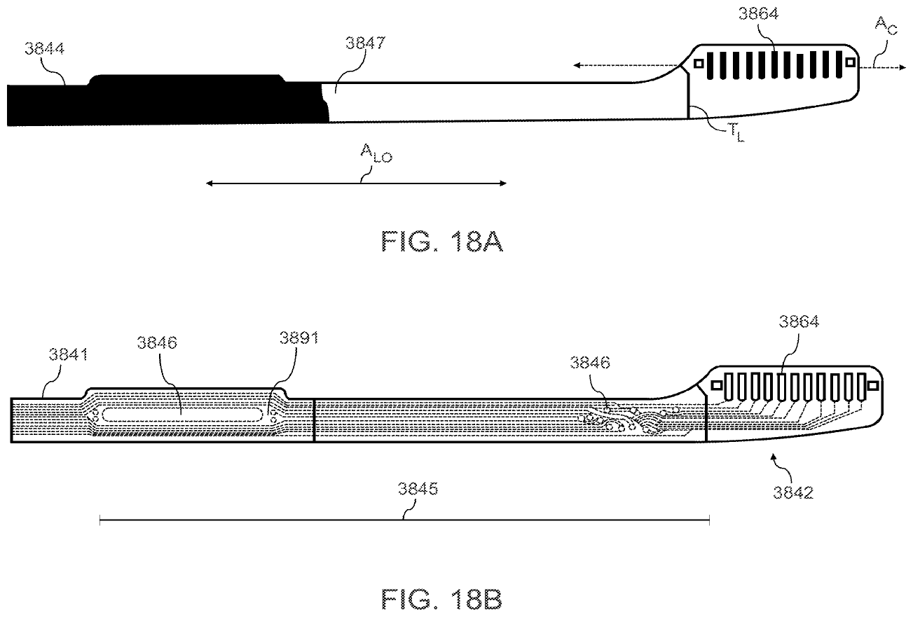

Referring still to FIGS. 8 and 9, and also to FIG. 12, In some embodiments, the proximal segment 2861 of the first layer 2860 includes a proximal coupling interface 2863. The proximal coupling interface 2863 includes a first set of electrically conductive contacts (e.g., electrically conductive contacts 3864 as depicted in FIG. 18A). The proximal segment 2861 of the first layer 2860 in the proximal end portion 2842 of the sensor cable 2840 is free of the first set of electrical traces 2890. In other words, the proximal segment 2861 does not contain any electrical traces and, therefore, defines an electrically-nonconductive region that extends across the width (e.g., along the lateral axis ALA) of the first layer 2860 between the first set of electrical traces 2890 in the first layer 2860 and the proximal coupling interface 2863. However, the proximal second layer 2870 is coupled to the proximal segment 2861 and contains a second set of electrical traces 2896. The second set of electrical traces 2896 are communicatively coupled between the electrically conductive contacts of the proximal coupling interface 2863 of the first layer 2860 and the first set of electrical traces 2890. In other words, during operation, an output signal from the force sensor unit can be transmitted proximally along the first set of electrical traces 2890 within the first layer 2860 to a proximal transfer portion 2845 where the first set of electrical traces 2890 terminate and the output signal can be communicated to a second set of electrical traces 2896 and on to the circuit board via the proximal coupling interface 2863.

As depicted, the proximal transfer portion 2845 is positioned between the proximal end portion 2842 and the middle portion 2841 of the sensor cable 2840. The proximal transfer portion 2845 includes a set of vias 2846. Each via 2846 can, for example, be an electrically conductive element or structure that is inserted or formed in/through two or more adjacent layers of the sensor cable 2840. Accordingly, each via 2846 is an electrical connection between the first set of electrical traces 2890 in the first layer 2860 and the second set of electrical traces 2896 in the proximal second layer 2870. Said another way, the set of vias 2846 within the proximal transfer portion 2845 are configured to communicatively couple the first set of electrical traces 2890 in the first layer 2860 in the middle portion 2841 to the second set of electrical traces 2896 in the proximal second layer 2870 in the proximal end portion 2842 of the sensor cable 2840. In some embodiments, the proximal transfer portion 2845 can facilitate a rearrangement of the electrical traces to establish a trace arrangement in conformity with an arrangement of the electrical contacts on the circuit board.

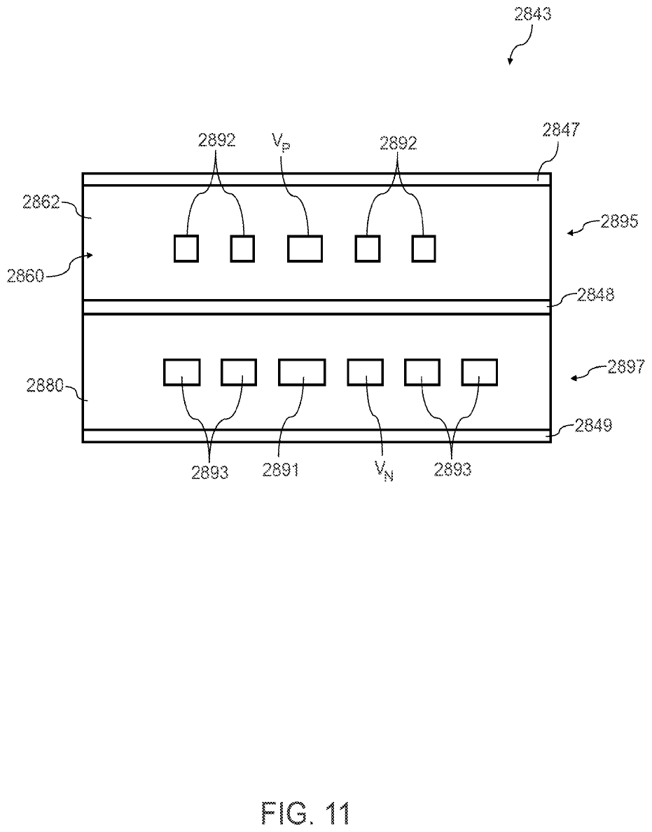

As depicted in FIGS. 8-12, in some embodiments, the sensor cable 2840 includes a first electrically insulative layer 2847, an electrically insulative base 2848, and a second electrically insulative layer 2849. The first electrically insulative layer 2847 is on the distal segment 2862 of the first layer 2860 (as illustrated in FIG. 11) and the middle portion 2841 of the sensor cable 2840 (as illustrated in FIGS. 10A-10B). The electrically insulative base 2848 extends between the distal end portion 2843 and the proximal end portion 2842 of the sensor cable 2840. The second electrically insulative layer 2849 on the distal second layer 2880, the middle portion 2841 of the sensor cable 2840 and the proximal second layer 2870, as depicted in FIGS. 8 and 9. In other words, the second electrically insulative layer 2849 extends along the entirety of the longitudinal length of the sensor cable 2840.

As depicted in FIGS. 8, 9, and 12, the first electrically insulative layer 2847 is absent from the proximal segment 2861 of the first layer 2860. In other words, because the first set of electrical traces 2890 terminate in the proximal transfer portion 2845 such that the proximal segment 2861 of the first layer 2860 does not contain electrical traces, it is not necessary to electrically insulate the proximal segment 2861. Therefore, the first electrically insulative layer 2847 can be terminated at the junction of the proximal transfer portion 2845 and the proximal end portion 2842 of the sensor cable 2840. By prohibiting the extension of the first electrically insulative layer 2847 onto the proximal segment 2861, the proximal segment 2861 can be maintained in a neutral orientation when the proximal coupling interface 2863 is coupled to the circuit board. Said another way, the absence of the first electrically insulative layer 2847 on the proximal segment 2861 precludes a necessity to flex or deform the first layer 2860 in order to bring the set of electrical contacts of the proximal coupling interface 2863 into contact with the circuit board. As the thickness of the first electrically insulative layer 2847 does not exist between the proximal segment 2861 and the circuit board, the planar nature of the proximal segment 2861 can be maintained and a separation force between the various layers of the sensor cable 2840 that would otherwise result from the bending/flexing is reduced or eliminated. Similarly stated, the proximal segment 2861 is coupled flush to the surface of the circuit board without any residual spring-back force that could otherwise be present if a step or discontinuity was present between the proximal segment 2861 and the circuit board. This, in turn, reduces or eliminates delamination of the sensor cable 2840 at the coupling with the circuit board, thereby, facilitating post procedure processing of the instrument.

FIG. 10A is a schematic cross-sectional view of the middle portion 2841 of the sensor cable 2840 taken along line X₁-X₁. As depicted, the first set of electrical traces 2890 includes positive traces 2892 and negative traces 2893 in the middle portion 2841 of the sensor cable 2840. In some embodiments, each positive trace 2892 is configured to carry a signal at a positive electrical potential. Similarly, in some embodiments, each negative trace is configured to carry a signal at a negative electrical potential. In some embodiments, the positive traces 2892 are the traces that are electrically coupled to distal portions (e.g., primary distal bridge-circuit combination 3832 and secondary distal bridge-circuit combination 3836 described below) of the strain sensor, while the negative traces 2893 are the traces that are electrically coupled to proximal portion (e.g., primary proximal bridge-circuit combination 3834 and secondary proximal bridge-circuit combination 3838 described below) of the strain sensor. Each positive trace 2892 has a first cross-sectional area CA₁. Each negative trace 2893 has a second cross-sectional area CA₂. In some embodiments, the first cross-sectional area CA₁ is smaller than the second cross-sectional area CA₂. Accordingly, the positive traces 2892 have a higher resistive value than the negative traces 2893. For example, the positive traces 2892 can have a resistance that is less than 20 ohms (e.g., less than 17 ohms), while the negative traces 2893 can have a resistance that is less than 10 ohms (e.g., less than 7 ohms).

In some embodiments, a first maximal resistance limit determines a minimal first cross-sectional area CAI of the positive traces 2892. Similarly, a second maximal resistance limit determines a minimal second cross-sectional area CA₂ of the negative traces 2893. In other words, while it may be otherwise desirable to minimize the cross-sectional areas of the first set of electrical traces 2890 to minimize the width and/or thickness of the sensor cable 2840 (due to size constraints within the instrument shaft and the desirability of flexibility of the sensor cable 2840), the maximal resistance limits establish boundaries at which further reductions in the cross-sectional areas will negatively affect the transmission of the signals. Relatedly, in some embodiments, a maximal sensor cable width W₁ defines a maximal combined cross-sectional area of each of the positive traces 2892 and each of the negative traces 2893 in the middle portion 2841 of the sensor cable 2840. The maximal sensor cable width W₁ is defined at least in part by a passage clearance (e.g., passage clearance W₂ as depicted in FIG. 7) of the instrument shaft. Said another way, while it may be otherwise desirable to maximize the cross-sectional areas of the first set of electrical traces 2890 to decrease the electrical resistance, size constraints within the instrument shaft establish an upper boundary to the sensor cable width. Accordingly, the maximal resistance limits establish a lower limit on the cross-sectional areas of the traces and, thus, the corresponding width and thickness of the sensor cable 2840, while the size constraints imposed by the instrument establish an upper limit on the width of the sensor cable 2840 and, thus, the cross-sectional areas of the traces. It should be appreciated that the positive traces 2892 and the negative traces 2893 can be tied to different circuity on the circuit board, which can result in differing impacts of interference.

Referring again to FIGS. 8 and 9, and also to FIG. 11, in some embodiments, the sensor cable includes a distal transfer portion 2850. The distal transfer portion 2850 is positioned between the middle portion 2841 and the distal end portion 2843 of the sensor cable 2840. The distal transfer portion 2850 includes a set of vias 2846. Each via 2846 can, for example, be an electrically conductive element or structure that is inserted or formed in/through two or more adjacent layers of the sensor cable 2840. Accordingly, each via 2846 is an electrical connection between a first portion of the first set of electrical traces 2890 in the first layer 2860 and a third set of electrical traces 2897 in the distal second layer 2880. Said another way, the set of vias 2846 within the distal transfer portion 2850 are configured to communicatively couple the first portion of the first set of electrical traces 2890 in the first layer 2860 in the middle portion 2841 to the third set of electrical traces 2897 in the distal second layer 2880 in the distal end portion 2843 of the sensor cable 2840.

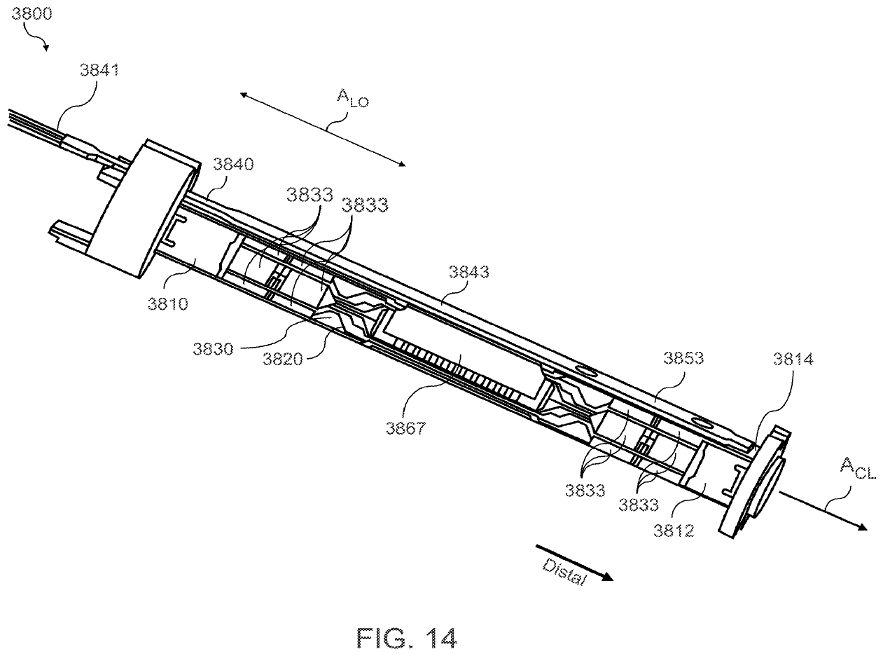

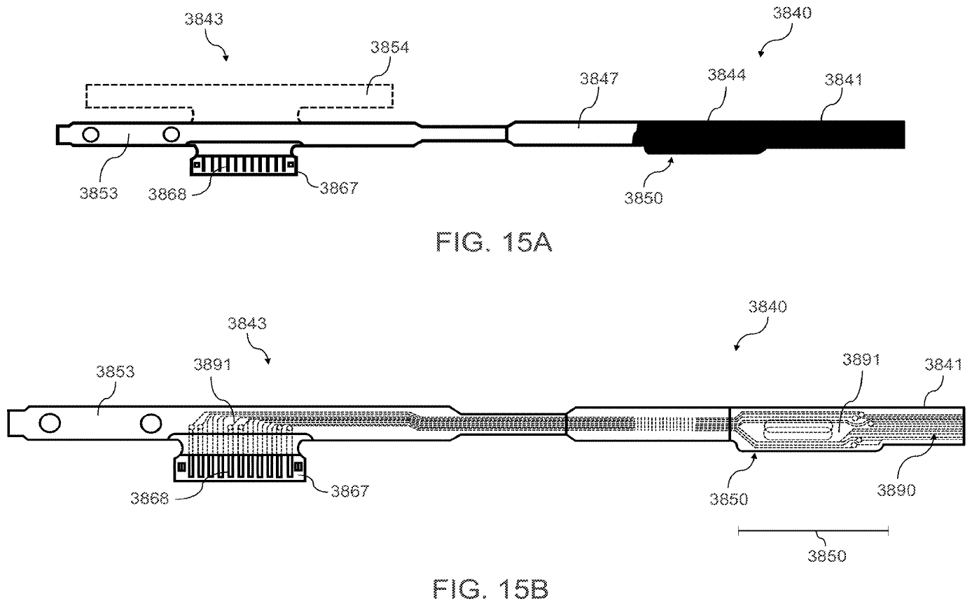

In some embodiments, the distal segment 2862 of the first layer 2860 in the distal end portion 2843 of the sensor cable 2840 defines a distal coupling interface 2867. The distal coupling interface 2867 includes a second set of conductive contacts (e.g., electrically conductive contacts 3868 as depicted in FIG. 15A) coupled to the force sensor unit (e.g., force sensor unit 3400 as depicted in FIG. 14). A second portion 2895 of the first set of electrical traces 2890 are communicatively coupled to the distal coupling interface 2867 in the first layer 2860 in the distal end portion 2843. In contrast to the proximal segment 2861, the distal segment 2862 includes electrical traces and is thus overlaid with the first electrically insulative layer 2847. The third set of electrical traces 2897 in the distal second layer 2880 is coupled to the distal coupling interface 2867.

In some embodiments, a linear arrangement of the second set of conductive contacts of the distal coupling interface 2867 establishes an initial configuration of the third set of electrical traces 2897 and the second portion 2895 of the first set of electrical traces 2890. Being a cross-sectional view of the distal end portion 2843, FIG. 11 depicts the initial configuration of the electrical traces in the distal end portion. As depicted, in some embodiments, the positive traces 2892 are positioned within the distal segment 2862 of the first layer 2860. The negative traces 2893 and the electrical ground trace 2891 are positioned within the distal second layer 2880. In some embodiments, the third set of electrical traces 2897 and the second portion 2895 of the first set of electrical traces 2890 are rearranged within the distal transfer portion 2850. The rearrangement establishes all electrical traces of the first set of electrical traces 2890 in a side-by-side, planar configuration within the first layer 2860 through the middle portion 2841 of the sensor cable 2840. In other words, the distal transfer portion 2850 facilitates the transition of the electrical traces between the arrangement depicted in FIG. 11 and the arrangements depicted in FIG. 10A or 10B.

As depicted in FIG. 10A, in some embodiments, the middle portion 2841 of the sensor cable 2840 includes a first lateral side region 2851 and a second lateral side region 2852 separated by the electrical ground trace 2891. In some embodiments, the positive traces 2892 are positioned within the first lateral side region 2851. Similarly, the negative traces 2893 are positioned within the second lateral side region 2852. Therefore, the positive traces 2892 are separated from the negative traces 2893 by the electrical ground trace 2891. Accordingly, electromagnetic interference between the positive traces 2892 and negative traces 2893 can be mitigated by the electrical ground trace 2891 positioned therebetween.

Like FIG. 10A, FIG. 10B is also a schematic cross-sectional view of the middle portion 2841 of the sensor cable 2840 but with the first set of electrical traces 2890 positioned in a different arrangement. As depicted in FIG. 10B, the first set of electrical traces 2890 includes at least one positive trace 2892 and at least one negative trace 2893 in the middle portion 2841 of the sensor cable 2840. The positive trace 2892 and the negative trace 2893 are arranged in a positive-negative pairing PN₁. For example, as depicted, the first set of electrical traces 2890 can, in some embodiments, include four positive-negative pairings PN₁, PN₂, PN₃, PN₄. As is more fully described below, each of the four positive-negative pairings can correspond to one of four bridge circuits of the strain sensor. When arranged as a positive-negative pairing, an induced current in positive trace 2892 will be substantially equal to an induced current in the adjacent negative trace 2893. This equalization of the induced currents results in the canceling out of the effects of the electromagnetic interference on the signals transmitted by the positive-negative pairing of electrical traces. In other words, since the induced current in each of the electrical traces of the positive-negative pairing has substantially the same value, the voltage of the output signals delivered to the circuit board may have a greater magnitude, but the increase in voltage magnitude does not affect the voltage differential, and thus the indications of strain.

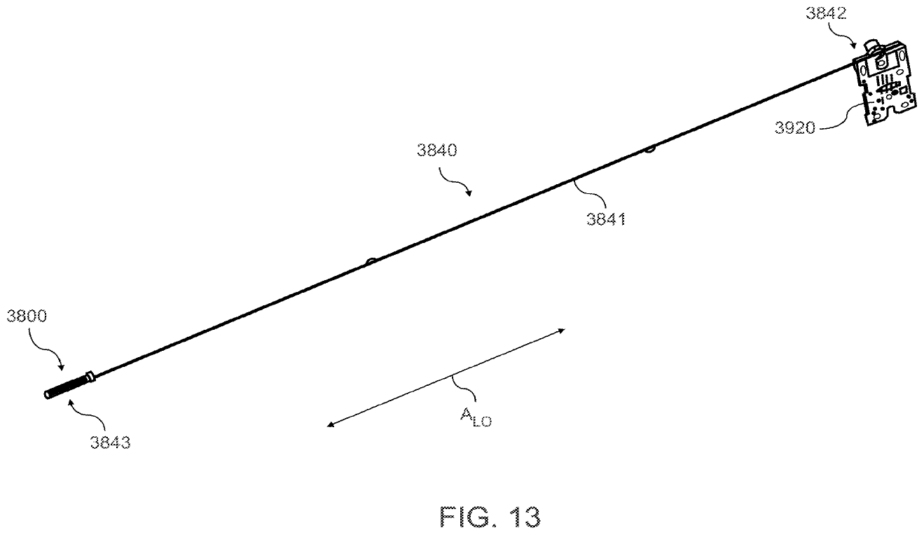

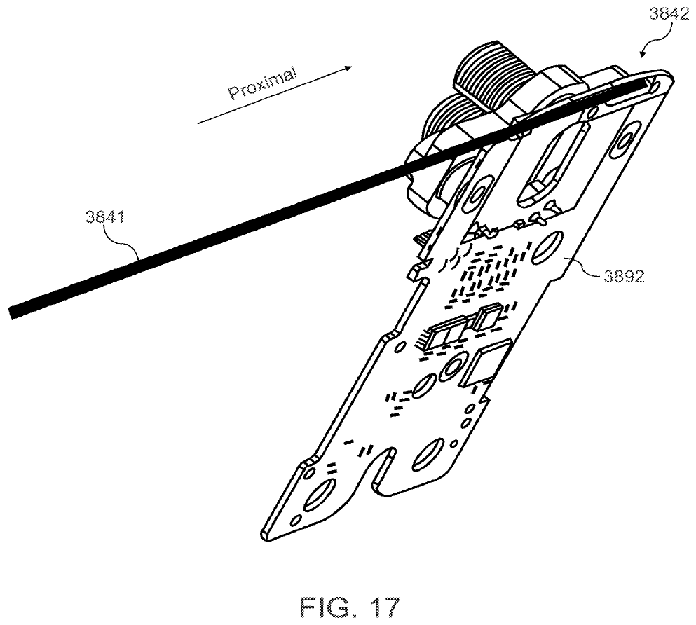

FIGS. 13-20C depict various view of aspects of a force sensor unit 3800 coupled to a circuit board 3920 via a sensor cable 3840 for use with a force sensing medical instrument, such as instrument 1400 described herein. In some embodiments, the force sensor unit 3800, the circuit board 3920, the sensor cable 3840, and/or any of the components thereof are optionally parts of a surgical system that performs surgical procedures. The surgical system may include a manipulator unit, a series of kinematic linkages, a series of cannulas, or the like. The force sensing medical instrument can include an instrument shaft coupled to a proximal mechanical structure, the force sensor unit 3800 coupled to the instrument shaft, and an end effector coupled to the force sensor unit 3800 via a wrist assembly as previously described. The force sensor unit 3800, the circuit board 3920, and/or the sensor cable 3840 (and any of the force sensor units, circuit boards, and/or sensor cables described herein) can be used in any suitable surgical system, such as the MIRS system 1000 shown and described above to mitigate the effects of electromagnetic interference when the instrument is exposed to an electrical field.

As depicted, the force sensor unit 3800 includes a beam 3810. The beam 3810 is a resiliently deflectable beam configured to bend or deflect in response to a load applied to a distal end portion of the instrument. A strain sensor 3830 is mounted on a first face 3812 (e.g., a lateral face) of the beam 3810 to sense strain that results from beam 3810 deflecting. The first face 3812 extends along a longitudinal axis A_{LO} and a lateral axis A_{LA} (FIG. 5) of the beam 3810. The beam 3810 can, for example, couple the distal end portion of the instrument (e.g., distal end portion 1402 (FIG. 5)) to the shaft of the instrument (e.g., shaft 1410 (FIG. 5)) in a cantilevered configuration anchored at the proximal end portion of the beam 3810.

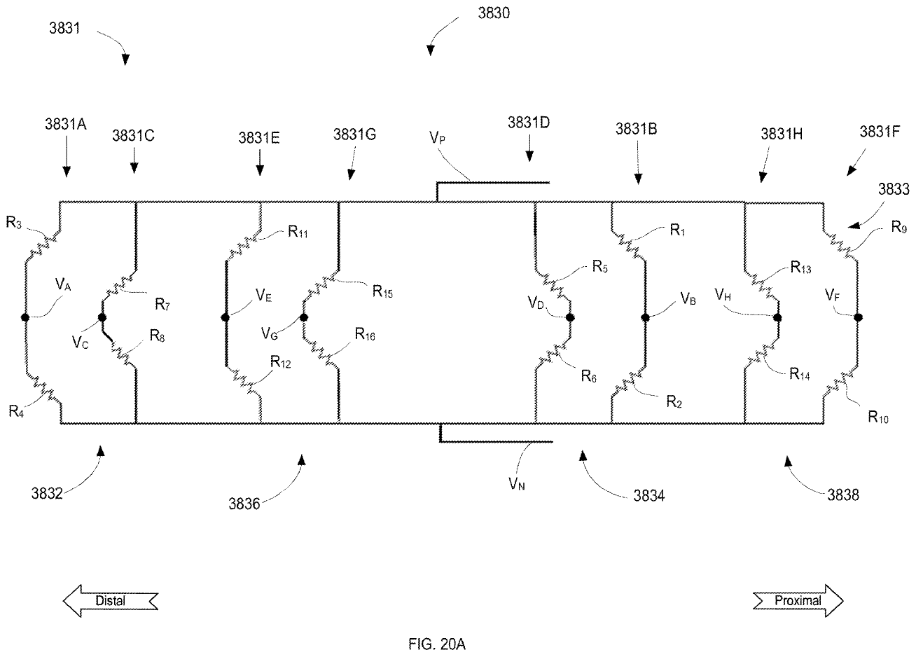

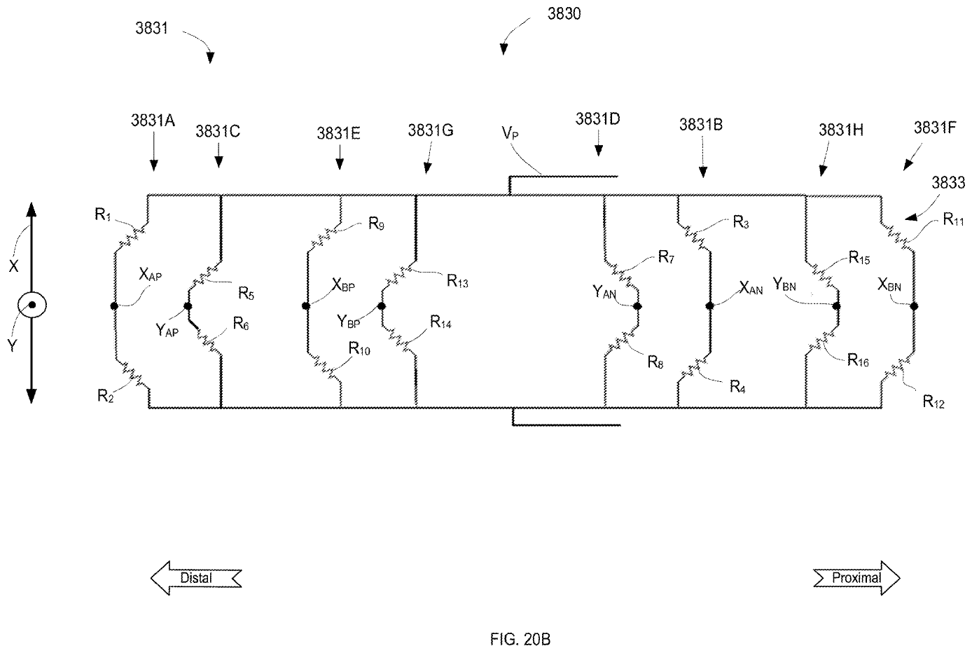

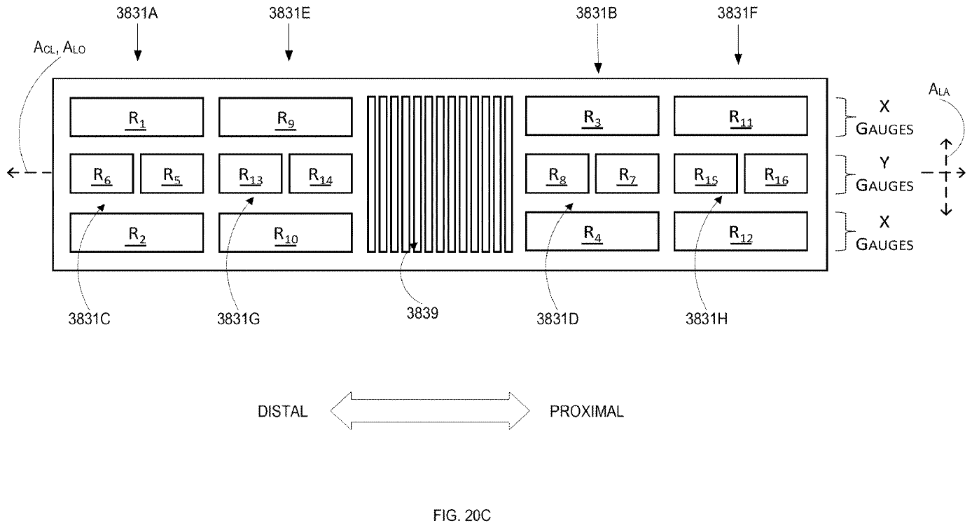

The strain sensor 3830 is optionally made of one or more electrical strain sensing circuits (e.g., half-bridge circuits 3831 (see e.g., FIGS. 20A-20C)), and other strain sensor configurations are contemplated (e.g., piezoelectric sensors, and the like). As described herein, each half-bridge circuit 3831 (and also each strain sensor) includes one or more strain gauges 3833 (e.g., tension strain gauge resistor(s) or compression strain gauge resistor(s)). It should be appreciated that the beam 3810 can include any number of strain sensors 3830 in various arrangements. In some embodiments, the beam 3810 includes a single strain sensor 3830 that includes multiple split half-bridge circuits 3831, with each split, half-bridge circuit 3831 having at least two strain gauges 3833.

During certain operations, the beam 3810 can be capacitively coupled to the strain sensor 3830 when exposed to an electrical field, the orthogonal distance between the first face 3812 of the beam 3810 and the strain gauges 3833 of the strain sensor 3830 can affect a current induced in the strain gauges 3833. When the distance between the strain gauges 3833 and the first face 3812 is uniform, the induced current in each of the strain gauges 3833 is substantially equal to the induced current in each other strain gauge 3833. This equalization of the induced currents results in the canceling out of the effects of the electromagnetic interference in the output of the strain sensor 3830. In other words, since the induced current in each of the strain gauges 3833 has substantially the same value, the voltage of the output signals may have a greater magnitude, but the increase in voltage magnitude does not affect the voltage differential, and thus the indications of strain. However, variations in the flatness of the first face 3812 and/or the thickness of an adhesive that couples the strain gauges 3833 to the beam 3810, can result in a lack of uniformity in the distance between the strain gauges 3833 and the first face 3812 and corresponding variations in the induced currents, which, in turn, manifest in the output signals of the strain sensor 3830 as electromagnetic interference. Therefore, the force sensor unit 3800 disclosed herein, in some embodiments, utilizes an electrically conductive layer and an electrically insulative layer to facilitate uniform capacitive coupling when the force sensor unit 3800 is exposed to an electrical field. Further details regarding the uniform capacitive coupling of the force sensor unit 3800 are provided in U.S. Provisional Ser. No. 63/425,518 , filed Nov. 15, 2022, the disclosure of which is incorporated herein by reference for all purposes.

In some embodiments, the strain sensor 3830 includes bridge circuits formed from pairs of half-bridge circuits 3831, a set of electrical pads (e.g., contacts, tap points, or pickup points), and an electrical trace structure 3820. The bridge circuit (e.g., a set of split-bridge circuits distributed along the longitudinal axis A_{LO} of the beam 3810) includes a set of strain gauges 3833 that are over (e.g., formed on) the electrically insulative layer. The bridge circuit 3831 has a uniform separation distance from the electrically conductive layer. The electrical trace structure 3820 is electrically coupled between the sensor cable 3840 and the electrical pads. Therefore, the electrical trace structure 3820 can provide an input voltage to the bridge circuit and can transmit an output signal indicative of strain to the sensor cable 3840.

In some embodiments, the electrical trace structure 3820 includes an input trace and one or more measurement traces (e.g., signal traces). The input trace is configured to deliver an input voltage (e.g., an excitation voltage) from the sensor cable 3840 to one or more split half-bridge circuits 3831. The measurement trace is configured to deliver an output signal from the split half-bridge circuit 3831 to the sensor cable 3840. For example, for each bridge circuit, a first half-bridge circuit 3831 can deliver an output signal to a positive electrical trace of the sensor cable 3840 and a second half-bridge circuit 3831 can deliver an output signal to a negative electrical trace of the sensor cable 3840.

As depicted in FIG. 13, the sensor cable 3840 includes a middle portion 3841 that is between a proximal end portion 3842 and a distal end portion 3843. The sensor cable 3840 includes a longitudinal axis A_{LO} that extends between the proximal end portion 3842 and the distal end portion 3843. As depicted in FIGS. 15B and 18B, the middle portion 3841 of the sensor cable 3840 includes a first set of electrical traces 3890. The first set of electrical traces 3890 can be communicatively coupled between a force sensor unit 3800 that is configured to measure a force affecting the instrument and the circuit board 3920 that is configured to receive an output from the force sensor unit 3800. The first set of electrical traces 3890 includes an electrical ground trace 3891. The electrical ground trace can electrically ground the force sensor unit 3800 to the circuit board 3920. In some embodiments, the electrical ground trace is electrically coupled to the beam 3810. In some embodiments, the force sensor unit 3800 can be electrically grounded to the circuit board by the drive cables, as described in U.S. Provisional Ser. No. 63/425,524, filed Nov. 15, 2022, the disclosure of which is incorporated herein by reference for all purposes. In some embodiments, the sensor cable 3840 includes an electrical shield 3844 that surrounds the middle portion 3841 of the sensor cable 3840 and is communicatively coupled to the electrical ground trace 3891. In some embodiments, the electrical shield 3844 extends between a proximal transfer portion 3845 and a distal transfer portion 3850 of the sensor cable 3840. The sensor cable 3840 can include any of the elements and features described herein with reference to sensor cable 2840 or sensor cable 1840.

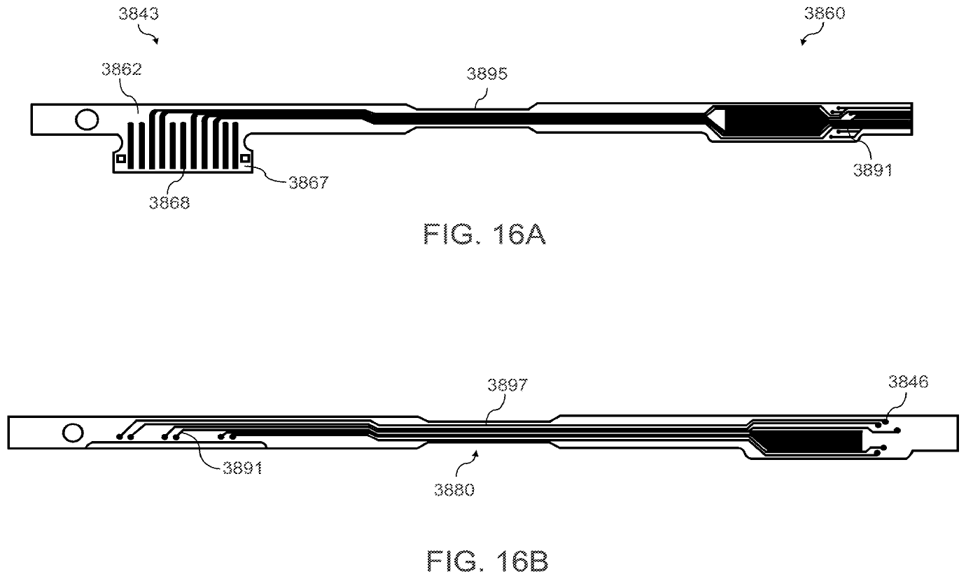

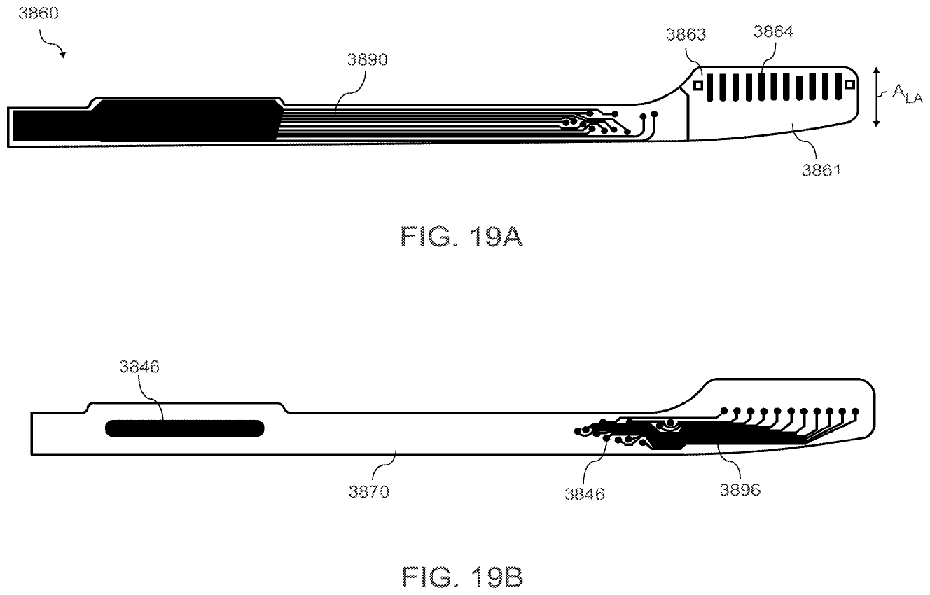

In some embodiments the sensor cable 3840 includes a first layer 3860 (e.g., FIGS. 16A and 19A), a proximal second layer 3870 (FIG. 19B), and a distal second layer 3880 (FIG. 16B). The first layer 3860 extends between the distal end portion 3843 of the sensor cable 3840 and the proximal end portion 3842 of the sensor cable 3840. The first layer 3860 includes a proximal segment 3861 that is within the proximal end portion 3842 of the sensor cable 3840. The first layer 3860 also includes a distal segment 3862 that is within the distal end portion 3843 of the sensor cable 3840. In other words, the first layer 3860 extends along the entirety of the sensor cable 3840.

In some embodiments, the proximal second layer 3870 extends parallel to the proximal segment 3861 of the first layer 3860 within the proximal end portion 3842 of the sensor cable 3840. FIG. 18B depicts the proximal second layer 3870 extending parallel to the proximal segment 3861 of the first layer 3860. However, FIGS. 19A and 19B depict the layers separated in the interest of clarity, with FIG. 19A depicting a portion of the first layer 3860, and FIG. 19B depicting the proximal second layer 3870. Similarly, in some embodiments, the distal second layer 3880 extends parallel to the distal segment 3862 of the first layer 3860 within the distal end portion 3843 of the sensor cable 3840. FIG. 15B depicts the distal second layer 3880 extending parallel to the distal segment 3862 of the first layer 3860. However, FIGS. 16A and 16B depict the layers separated in the interest of clarity, with FIG. 16A depicting a portion of the first layer 3860, and FIG. 16B depicting the distal second layer 3880. In some embodiments, the middle portion 3841 of the sensor cable 3840 is free of the proximal second layer 3870 and the distal second layer 3880. In such an embodiment, the first set of electrical traces 3890 can extend in a side-by-side planar configuration within the first layer 3860 through the middle portion 3841 of the sensor cable 3840. Said another way, neither the proximal second layer 3870 nor the distal second layer 3880 extend the entirety of the sensor cable 3840.

In some embodiments, the proximal segment 3861 of the first layer 3860 includes a proximal coupling interface 3863. The proximal coupling interface 3863 includes a first set of electrically conductive contacts 3864. In some embodiments, such as depicted in FIG. 18A, the first set of electrically conductive contacts 3864 are arranged along a contact axis A_{C} that is parallel to the longitudinal axis A_{LO} of the sensor cable 3840. In some embodiments, the proximal coupling interface 2863 is an anisotropic conductive film coupling. In some embodiments, the proximal coupling interface 2863 is a moisture-proof connector.

The proximal segment 3861 of the first layer 3860 in the proximal end portion 3842 of the sensor cable 3840 is free of the first set of electrical traces 3890. In other words, the proximal segment 3861 does not contain any electrical traces and, therefore, defines an electrically-nonconductive region that extends across the width (e.g., along the lateral axis A_{LA}) of the first layer 3860 between the first set of electrical traces 3890 in the first layer 3860 and the proximal coupling interface 3863. However, the proximal second layer 3870 is coupled to the proximal segment 3861 and contains a second set of electrical traces 3896. The second set of electrical traces 3896 are communicatively coupled between the electrically conductive contacts 3864 of the proximal coupling interface 3863 of the first layer 3860 and the first set of electrical traces 3890. In other words, during operation, an output signal from the force sensor unit 3800 can be transmitted proximally along the first set of electrical traces 3890 within the first layer 3860 to the proximal transfer portion 3845 where the first set of electrical traces 3890 terminate and the output signal can be communicated to a second set of electrical traces 3896 and on to the circuit board 3920 via the proximal coupling interface 3863.

As depicted in FIG. 18B, the proximal transfer portion 3845 is positioned between the proximal end portion 3842 and the middle portion 3841 of the sensor cable 3840. The proximal transfer portion 3845 includes a set of vias 3846 as described herein. The set of vias 3846 within the proximal transfer portion 3845 are configured to communicatively couple the first set of electrical traces 3890 in the first layer 3860 to the second set of electrical traces 3896 in the proximal second layer 3870 in the proximal end portion 3842 of the sensor cable 3840. In some embodiments, the proximal transfer portion 3845 can facilitate a rearrangement of the electrical traces to establish a trace arrangement in conformity with an arrangement of the electrical contacts on the circuit board.

As depicted in FIGS. 15A and 18A, in some embodiments, the sensor cable 3840 includes a first electrically insulative layer 3847, an electrically insulative base (not shown), and a second electrically insulative layer (not shown). The first electrically insulative layer 3847 is on the distal segment 3862 of the first layer 3860 and the middle portion 3841 of the sensor cable 3840 (as illustrated in FIGS. 10A-10B). The first electrically insulative layer 3847 is absent from the proximal segment 3861 of the first layer 3860. In other words, because the first set of electrical traces 3890 terminate in the proximal transfer portion 3845 such that the proximal segment 3861 of the first layer 3860 does not contain electrical traces, it is not necessary to electrically insulate the proximal segment 3861. Therefore, the first electrically insulative layer 3847 can be terminated at the junction of the proximal transfer portion 3845 and the proximal end portion 3842 of the sensor cable 3840, as indicated by the termination line T_{L}. By prohibiting the extension of the first electrically insulative layer 3847 onto the proximal segment 3861, the proximal segment 3861 can be maintained in a neutral orientation when the proximal coupling interface 3863 is coupled to the circuit board 3920. Said another way, the absence of the first electrically insulative layer 3847 on the proximal segment 3861 precludes a necessity to flex or deform the first layer 3860 in order to bring the set of electrically conductive contacts 3864 into contact with the circuit board 3920. As the thickness of the first electrically insulative layer 3847 does not exist between the proximal segment 3861 and the circuit board, the planar nature of the proximal segment 3861 can be maintained and a separation force between the various layers of the sensor cable 3840 that would otherwise result from the bending/flexing is reduced or eliminated. Similarly stated, the proximal segment 3861 is coupled flush to the surface of the circuit board without any residual spring-back force that could otherwise be present if a step or discontinuity was present between the proximal segment 3861 and the circuit board. This, in turn, reduces or eliminates delamination of the sensor cable 3840 at the coupling with the circuit board, thereby, facilitating post procedure processing of the instrument.

Referring again to FIGS. 15B-16B, in some embodiments, the sensor cable includes a distal transfer portion 3850. The distal transfer portion 3850 is positioned between the middle portion 3841 and the distal end portion 3843 of the sensor cable 3840. The distal transfer portion 3850 includes a set of vias 3846. Accordingly, each via 3846 is an electrical connection between a first portion of the first set of electrical traces 3890 in the first layer 3860 and a third set of electrical traces 3897 in the distal second layer 3880.

In some embodiments, the distal segment 3862 of the first layer 3860 in the distal end portion 3843 of the sensor cable 3840 defines a distal coupling interface 3867. The distal coupling interface 3867 includes a second set of electrically conductive contacts 3868 coupled to the force sensor unit 3800 as depicted in FIG. 14. As depicted in FIG. 16A, a second portion 3895 of the first set of electrical traces 3890 are communicatively coupled to the distal coupling interface 3867 in the first layer 3860 in the distal end portion 3843. In contrast to the proximal segment 3861, the distal segment 3862 includes electrical traces and is thus overlaid with the first electrically insulative layer 3847. The third set of electrical traces 3897 in the distal second layer 3880 is coupled to the distal coupling interface 3867. In some embodiments, the distal coupling interface 3867 is an anisotropic conductive film coupling. In some embodiments, the distal coupling interface 3867 is a moisture-proof connector. In some embodiments, the distal coupling interface 3867 can mechanically and electrically couple the sensor cable to the force sensor unit (e.g., via ACF).

In some embodiments, the distal coupling interface 3867 is configured to be positioned orthogonally to the remainder of the distal end portion 3843 of the sensor cable 3840 on a condition that the second set of electrically conductive contacts 3868 are electrically coupled to the force sensor unit 3800 as depicted in FIG. 14. Said another way, the distal coupling interface 3867 is configured to be folded relative to the remainder of the distal end portion 3843 of the sensor cable 3840 on a condition that the distal coupling interface 3867 is mechanically coupled to the force sensor unit 3800. Accordingly, on the condition that the distal coupling interface 3867 is mechanically coupled to the force sensor unit 3800, the distal coupling interface 3867 can extend along a portion of the lateral face 3812 of the beam 3810 and be separated from the lateral face 3812 by the stain sensor 3830. On the same condition, the remainder of the distal end portion 3843 of the sensor cable 3840 can extend along a second face of the beam 3810. For example, in some embodiments, the remainder of the distal end portion 3843 can be mechanically coupled to a second face of the beam 3810 that is orthogonal to the lateral face 3812 as depicted in FIG. 14. In some embodiments, the remainder of the distal end portion 3843 can be mechanically coupled to a second face of the beam 3810 that is substantially parallel to the lateral face 3812. Said yet another way, the distal coupling interface can be configured to be folded over and coupled to the force sensor unit 3800 on a first face of the beam while the remainder of the distal end portion 3843 is mechanically coupled to the second face of the beam 3810 that is orthogonal to the first face. With reference to FIG. 14, the distal coupling interface 3867 can be pre-folded (e.g., folded prior to being coupled to the force sensor unit and/or during manufacturing of the sensor cable). In some embodiments, the magnitude of the pre-fold corresponds to an angle between the first face of the beam 3810 and the second face of the beam 3810. It should be appreciated that forming the distal coupling interface 3867 with a pre-fold relative to the remainder of the distal end portion 3843 of the sensor cable 3840 can facilitate the maintenance of the mechanical coupling between the distal coupling interface 3867 and the force sensor unit 3800.

In some embodiments, a linear arrangement of the second set of conductive contacts of the distal coupling interface 3867 establishes an initial configuration of the third set of electrical traces 3897 and the second portion 3895 of the first set of electrical traces 3890. For example, in some embodiments, the positive traces 3892 are positioned within the distal segment 3862 of the first layer 3860. The negative traces 3893 and the electrical ground trace 3891 are positioned within the distal second layer 3880. In some embodiments, the third set of electrical traces 3897 and the second portion 3895 of the first set of electrical traces 3890 are rearranged within the distal transfer portion 3850. The rearrangement establishes all electrical traces of the first set of electrical traces 3890 in a side-by-side, planar configuration within the first layer 3860 through the middle portion 3841 of the sensor cable 3840. In other words, the distal transfer portion 3850 facilitates the transition of the electrical traces between a first arrangement in the distal end portion 3843 and a second arrangement in the middle portion 3841. For example, in some embodiments, positive traces can be arranged in one portion of the sensor cable 3840 while negative traces are arranged in another portion of the sensor cable 3840, with the electrical ground trace 3891 disposed therebetween. Alternatively, positive and negative traces of the first set of electrical traces 3890 can be arranged in a number of positive-negative pairings as described herein.

Referring again to FIGS. 14-15B, in some embodiments, the sensor cable 3840 includes a balancing portion 3853. The balancing portion 3853 can extend distally from the distal end portion 3843 of the sensor cable 3840. The balancing portion 3853 can have a stiffness that corresponds to (e.g., is substantially equal to) a stiffness of the distal end portion 3843 of the sensor cable 3840 but does not include any electrical traces. The balancing portion 3853 can facilitate a uniform increase in the stiffness of the beam 3810 that results from the coupling of the distal end portion 3843 to a second face 3814 of the beam 3810. In other words, the balancing portion can mitigate the effects of a local stiffness concentration that can be developed as a result of the coupling of the distal end portion 3843 to the beam 3810. Similarly, as depicted in FIG. 15A, in some embodiments, the sensor cable 3840 can include a stiffness-balancing tab 3854. The stiffness-balancing tab 3854 can be coupled to a third face (not shown) of the beam 3810. The stiffness-balancing tab 3854 can have a stiffness that corresponds to a stiffness of the strain sensor 3830 and have an absence of electrical traces. In additional embodiments, the stiffness-balancing tab 3054 can be extended onto a fourth face (not shown) of the beam 3810 such that an increase in beam stiffness resulting from the components coupled thereto is uniform about a neutral axis of the beam 3810.

FIGS. 20A and 20B are diagrammatic illustrations of configurations of the strain sensor 3830 depicted in FIG. 14, showing eight half-bridge circuits 3831A-3831H having a set of strain gauges 3833 (R₁-R₁₆) of a four full bridge circuit configuration. The eight half-bridge circuits 3831 include a first half-bridge circuit 3831A, a second half-bridge circuit 3831B, a third half-bridge circuit 3831C, a fourth half-bridge circuit 3831D, a fifth half-bridge circuit 3831E, a sixth half-bridge circuit 3831F, a seventh half-bridge circuit 3831G, and an eighth half-bridge circuit 3831H. In order to detect strain, an input voltage (eg, positive input voltage VP and negative input voltage V_{N}) is provided to the eight half-bridge circuits 3831(A-H), and an output voltage (e.g., V_{A}, V_{B}, V_{c}, V_{D}, V_{E}, V_{F}, V_{G}, and V_{H} (V_{A-H})) can then be measured for each of the eight half-bridge circuits 3831(A-H). Various combinations of the output voltages (V_{A-H}) may be employed by the controller to determine a magnitude of the force affecting the instrument based on the sensed strain.

As depicted in FIG. 20A, in some embodiments, the first half-bridge circuit 3831A and the third half-bridge circuit 3831C are arranged as a primary distal bridge-circuit combination 3832, while the second half-bridge circuit 3831B and the fourth half-bridge circuit 3831D are arranged as a primary proximal bridge-circuit combination 3834. Additionally, in some embodiments, the fifth half-bridge circuit 3831E and the seventh half-bridge circuit 3831G are arranged as a secondary distal bridge-circuit combination 3836, while the sixth half-bridge circuit 3831F and the eighth half-bridge circuit 3831H are arranged as the secondary proximal bridge-circuit combination 3838. An output of the secondary distal bridge-circuit combination 3836 is redundant to a corresponding output of the primary distal bridge-circuit combination 3832. Similarly, an output of the secondary proximal bridge-circuit combination 3838 is redundant to a corresponding output of the primary proximal bridge-circuit combination 3834. In other words, absent a sensor malfunction, the outputs of the secondary distal bridge-circuit combination 3836 and the secondary proximal bridge-circuit combination 3838 equal the outputs of the primary distal bridge-circuit combination 3832 and the primary proximal bridge-circuit combination 3834.

As depicted in FIG. 20A, the first half-bridge circuit 3831A can include the third strain gauge resistor (R₃) and the fourth strain gauge resistor (R₄). The third and fourth strain gauge resistors (R₃, R₄) can be positioned on opposite sides of a beam center axis A_{CL} (FIG. 14) (e.g., a longitudinal axis A_{LO} that is centered laterally on a lateral surface 3812 of the beam 3810) and equidistant from the center axis. For example, the third and fourth strain gauge resistors (R₃, R₄) can be positioned equidistant between the beam center axis A_{CL} and a side edge of the surface to which they are mounted. In some embodiments, the third and fourth strain gauge resistors (R₃, R₄) can be positioned at the same proximal position along the beam center axis A_{CL}. In some embodiments, the third and fourth strain gauge resistors (R₃, R₄) are both the same type of strain gauge resistor (e.g., are both tension strain gauge resistors).

As further depicted in FIG. 20A, the third half-bridge circuit 3831C can include the seventh strain gauge resistor (R₇) and the eighth strain gauge resistor (R₈). The seventh and eighth strain gauge resistors (R₇, R₈) are positioned in axial alignment with the beam center axis A_{CL}. In some embodiments, a portion of the eighth strain gauge resistor (R₈) is positioned axially between the portions of the seventh strain gauge resistor (R₇), and a portion of the seventh strain gauge resistor (R₇) is positioned axially between the portions of the eighth strain gauge resistor (R₈). In some embodiments, one of the seventh and eighth strain gauge resistors (R₇, R₈) is a tension strain gauge resistor while the other is a compression strain gauge resistor.

As depicted in FIG. 20A, the second half-bridge circuit 3831B can include the first strain gauge resistor (R₁) and the second strain gauge resistor (R₂). The first and second strain gauge resistors (R₁, R₂) can be positioned on opposite sides of the beam center axis A_{CL} and equidistant from the center axis. For example, the first and second strain gauge resistors (R₁, R₂) can be positioned equidistant between the beam center axis A_{CL} and a side edge of the surface to which they are mounted. In some embodiments, the first and second strain gauge resistors (R₁, R₂) can be positioned at the same proximal position along the beam center axis A_{CL}. In some embodiments, the first and second strain gauge resistors (R₁, R₂) are both the same type of strain gauge resistor (e.g., are both tension strain gauge resistors).

As further depicted in FIG. 20A, the fourth half-bridge circuit 3831D can include the fifth strain gauge resistor (R₅) and the sixth strain gauge resistor (R₆). The fifth and sixth strain gauge resistors (R₅, R₆) are positioned in axial alignment with the beam center axis A_{CL}. In some embodiments, a portion of the sixth strain gauge resistor (R₆) is positioned axially between the portions of the fifth strain gauge resistor (R₅), and a portion of the fifth strain gauge resistor (R₅) is positioned axially between the portions of the sixth strain gauge resistor (R₆). One of the fifth and sixth strain gauge resistors (R₅, R₆) is a tension strain gauge resistor while the other is a compression strain gauge resistor.

Referring again to FIG. 20A, as depicted, the fifth half-bridge circuit 3831E can include the eleventh strain gauge resistor (R₁₁) and the twelfth strain gauge resistor (R₁₂). The eleventh and twelfth strain gauge resistors (R₁₁, R₁₂) can be positioned on opposite sides of the beam center axis A_{CL} and equidistant from the center axis. For example, the eleventh and twelfth strain gauge resistors (R₁₁, R₁₂) can be positioned equidistant between the beam center axis A_{CL} and a side edge of the surface to which they are mounted. In some embodiments, the eleventh and twelfth strain gauge resistors (R₁₁, R₁₂) can be positioned at the same proximal position along the beam center axis A_{CL}. In some embodiments, the eleventh and twelfth strain gauge resistors (R₁₁, R₁₂) are both the same type of strain gauge resistor (e.g., are both tension strain gauge resistors). The fifth half-bridge circuit 3831E is positioned distally relative to the first half-bridge circuit 3831A.

As further depicted in FIG. 20A, the seventh half-bridge circuit 3831G can include the fifteenth strain gauge resistor (R₁₅) and the sixteenth strain gauge resistor (R₁₆). The fifteenth and sixteenth strain gauge resistors (R₁₅, R₁₆) are positioned in axial alignment with the beam center axis A_{CL}. In some embodiments, a portion of the fifteenth strain gauge resistor (R₁₅) is positioned axially between the portions of the sixteenth strain gauge resistor (R₁₆), and a portion of the sixteenth strain gauge resistor (R₁₆) is positioned axially between the portions of the fifteenth strain gauge resistor (R₁₅). One of the fifteenth and sixteenth strain gauge resistors (R₁₅, R₁₆) is a tension strain gauge resistor while the other is a compression strain gauge resistor. The seventh half-bridge circuit 3831G is positioned distally relative to the third half-bridge circuit 3831C.

As depicted in FIG. 20A, the sixth half-bridge circuit 3831F can include the 9^{th} strain gauge resistor (R₉) and the tenth strain gauge resistor (R₁₀). The ninth and tenth strain gauge resistors (R₉, R₁₀) can be positioned on opposite sides of the beam center axis Act and equidistant from the center axis. For example, the ninth and tenth strain gauge resistors (R₉, R₁₀) can be positioned equidistant between the beam center axis A_{CL} and a side edge of the surface to which they are mounted. In some embodiments, the ninth and tenth strain gauge resistors (R₉, R₁₀) can be positioned at the same proximal position along the beam center axis A_{CL}. In some embodiments, the ninth and tenth strain gauge resistors (R₉, R₁₀) are both the same type of strain gauge resistor (e.g., are both tension strain gauge resistors). The sixth half-bridge circuit 3831F is positioned distally relative to the second half-bridge circuit 3831B.

As further depicted in FIG. 20A, the eighth half-bridge circuit 3831H can include the thirteenth strain gauge resistor (R₁₃) and the fourteenth strain gauge resistor (R₁₄). The thirteenth and fourteenth strain gauge resistors (R₁₃, R₁₄) are positioned in axial alignment with the beam center axis A_{CL}. In some embodiments, a portion of the thirteenth strain gauge resistor (R₁₃) is positioned axially between the portions of the fourteenth strain gauge resistor (R₁₄), and a portion of the fourteenth strain gauge resistor (R₁₄) is positioned axially between the portions of the thirteenth strain gauge resistor (R₁₃). One of the thirteenth and fourteenth strain gauge resistors (R₁₃, R₁₄) is a tension strain gauge resistor while the other is a compression strain gauge resistor. The eighth half-bridge circuit 3831H is positioned distally relative to the fourth half-bridge circuit 3831D.

As depicted in FIGS. 20B and 20C, in some embodiments, the strain sensor includes a four full-bridge circuit arrangement with each full bridge circuit including two half-bridge circuits for a total of eight half-bridge circuits. The corresponding half-bridge circuits of each full bridge circuit are located on opposite end portions of the strain sensor (e.g., on a distal end portion and a proximal end portion) in contrast to the arrangement depicted in FIG. 20A. The first half-bridge circuit 3831A is positioned at the distal end portion 3815 of the beam 3810, while the second half-bridge circuit 3831B is positioned at the proximal end portion 3813 of the beam 3810. The sensor cable 2840, pads 3839, and/or anisotropic conductive film (ACF) can extend or be disposed therebetween (e.g., separating the first and second-half-bridges, the distal and proximal end portion half-bridges) as described in more detail below. The first half-bridge circuit 3831A and the second half-bridge circuit 3831B can be electrically coupled to form a first primary-full-bridge circuit. The first primary-full-bridge circuit can be configured to measure strain imparted along a first axis. The first axis can, for example, be lateral to the lateral face 3812 of the beam 3810 (e.g., in the direction of the lateral axis A_{LA}). In some embodiments, the first axis is an X-axis and the strain gauges of the first primary-full-bridge circuit can each be tension strain gauge resistors (e.g., to measure strain along the X-axis). In some embodiments, the tension strain gauge resistors described herein can have elongated portions aligned in parallel and coupled end-to-end to form a serpentine or snake-like configuration. The elongated portions of the tension gauge resistors can extend or be aligned parallel to the longitudinal axis A_{LO}.

As depicted, first half-bridge circuit 3831A can include the first strain gauge resistor (R₁) and the second strain gauge resistor (R₂). The first and second strain gauge resistors (R₁, R₂) can be positioned on opposite sides of the beam center axis A_{CL} and equidistant from the center axis. For example, the first and second strain gauge resistors (R₁, R₂) can be positioned equidistant between the beam center axis A_{CL} and a side edge of the surface to which they are mounted. In some embodiments, the first and second strain gauge resistors (R₁, R₂) can be positioned at the same distal position along the beam center axis A_{CL}. In some embodiments, the first and second strain gauge resistors (R₁, R₂) are both the same type of strain gauge resistor (e.g., are both tension strain gauge resistors).

As further depicted, the second half-bridge circuit 3831B can include the third strain gauge resistor (R₃) and the fourth strain gauge resistor (R₄). The third and fourth strain gauge resistors (R₃, R₄) can be positioned on opposite sides of a beam center axis A_{CL} (e.g., a longitudinal axis A_{LO} that is centered laterally on a lateral surface 3812 of the beam 3810) and equidistant from the center axis. For example, the third and fourth strain gauge resistors (R₃, R₄) can be positioned equidistant between the beam center axis A_{CL} and a side edge of the surface to which they are mounted. In some embodiments, the third and fourth strain gauge resistors (R₃, R₄) can be positioned at the same proximal position along the beam center axis A_{CL}. In some embodiments, the third and fourth strain gauge resistors (R₃, R₄) are both the same type of strain gauge resistor (e.g., are both tension strain gauge resistors).

As depicted in FIGS. 20B and 20C, in some embodiments, the third half-bridge circuit 3831C is positioned at the distal end portion 3815 of the beam 3810, while the fourth half-bridge circuit 3831D is positioned at the proximal end portion 3813 of the beam 3810. The third half-bridge circuit 3831C and the fourth half-bridge circuit 3831D can be electrically coupled to form a second primary-full-bridge circuit. The second primary-full-bridge circuit can be configured to measure strain imparted along a second axis orthogonal to the first axis (e.g., to measure strain along the second axis). The second axis can, for example, be normal to the lateral face 3812 of the beam 3810. In some embodiments, the second axis is a Y-axis and the strain gauges of the second primary-full-bridge circuit can be a combination of tension strain gauge resistors and compression strain gauge resistors. As discussed above with respect to the first primary full-bridge circuit, the tension strain gauge resistors described herein can have elongated portions aligned in parallel and coupled end-to-end to form a serpentine or snake-like configuration. The elongated portions of the tension gauge resistors can extend or be aligned parallel to the longitudinal axis A_{LO}. Similarly, the compression strain gauge resistors described herein can also have elongated portions aligned in parallel and coupled end-to-end to form a serpentine or snake-like configuration. However, the elongated portions of the compression gauge resistors described herein can extend or be aligned transverse to the longitudinal axis A_{LO} (e.g., parallel to the lateral axis A_{LA}).

As depicted, the third half-bridge circuit 3831C can include the fifth strain gauge resistor (R₅) and the sixth strain gauge resistor (R₆). The fifth and sixth strain gauge resistors (R₅, R₆) are positioned in axial alignment with the beam center axis A_{CL}. In some embodiments, a portion of the sixth strain gauge resistor (R₆) is positioned axially between the portions of the fifth strain gauge resistor (R₅), and/or a portion of the fifth strain gauge resistor (R₅) is positioned axially between the portions of the sixth strain gauge resistor (R₆). In some embodiments, one of the fifth and sixth strain gauge resistors (R₅, R₆) is a tension strain gauge resistor while the other is a compression strain gauge resistor.

As further depicted, the fourth half-bridge circuit 3831D can include the seventh strain gauge resistor (R₇) and the eighth strain gauge resistor (R₈). The seventh and eighth strain gauge resistors (R₇, R₈) are positioned in axial alignment with the beam center axis A_{CL}. In some embodiments, a portion of the eighth strain gauge resistor (R₈) is positioned axially between the portions of the seventh strain gauge resistor (R₇), and/or a portion of the seventh strain gauge resistor (R₇) is positioned axially between the portions of the eighth strain gauge resistor (R₈). In some embodiments, one of the seventh and eighth strain gauge resistors (R₇, R₈) is a tension strain gauge resistor while the other is a compression strain gauge resistor.

As depicted in FIGS. 20B and 20C, in some embodiments, the fifth half-bridge circuit 3831E is positioned at the distal end portion 3815 of the beam 3810, while the sixth half-bridge circuit 3831F is positioned at the proximal end portion 3813 of the beam 3810. The fifth half-bridge circuit 3831E and the sixth half-bridge circuit 3831F can be electrically coupled to form a first secondary-full-bridge circuit. The first secondary-full-bridge circuit can be configured to measure strain imparted along the first axis. In some embodiments, the first secondary-full-bridge circuit can each be tension strain gauge resistors.

As depicted, the fifth half-bridge circuit 3831E can include the ninth strain gauge resistor (R₉) and the tenth strain gauge resistor (R₁₀). The ninth and tenth strain gauge resistors (R₉, R₁₀) can be positioned on opposite sides of the beam center axis A_{CL} and equidistant from the center axis. For example, the ninth and tenth strain gauge resistors (R₉, R₁₀) can be positioned equidistant between the beam center axis A_{CL} and a side edge of the surface to which they are mounted. In some embodiments, the ninth and tenth strain gauge resistors (R₉, R₁₀) can be positioned at the same distal position along the beam center axis A_{CL}. In some embodiments, the ninth and tenth strain gauge resistors (R₉, R₁₀) are both the same type of strain gauge resistor (e.g., are both tension strain gauge resistors). The fifth half-bridge circuit 3831E can be positioned longitudinally between the first half-bridge circuit 3831A and the second half-bridge circuit 3831B (e.g., proximally relative to the first half-bridge circuit 3831a and distally relative to the second half-bridge circuit 3831B).

As further depicted, the sixth half-bridge circuit 3831F can include the eleventh strain gauge resistor (R₁₁) and the twelfth strain gauge resistor (R₁₂). The eleventh and twelfth strain gauge resistors (R₁₁, R₁₂) can be positioned on opposite sides of the beam center axis A_{CL} and equidistant from the center axis. For example, the eleventh and twelfth strain gauge resistors (R₁₁, R₁₂) can be positioned equidistant between the beam center axis A_{CL} and a side edge of the surface to which they are mounted. In some embodiments, the eleventh and twelfth strain gauge resistors (R₁₁, R₁₂) can be positioned at the same proximal position along the beam center axis A_{CL}. In some embodiments, the eleventh and twelfth strain gauge resistors (R₁₁, R₁₂) are both the same type of strain gauge resistor (e.g., are both tension strain gauge resistors). The sixth half-bridge circuit 3831F can be positioned proximally relative to the second half-bridge circuit 3831B.

As depicted in FIGS. 20B and 20C, in some embodiments, the seventh half-bridge circuit 3831G is positioned at the distal end portion 3815 of the beam 3810, while the eighth half-bridge circuit 3831H is positioned at the proximal end portion 3813 of the beam 3810. The seventh half-bridge circuit 3831G and the eighth half-bridge circuit 3831H can be electrically coupled to form a second secondary-full-bridge circuit. The second secondary-full-bridge circuit can be configured to measure strain imparted along the second axis. In some embodiments, the second secondary-full-bridge circuit can be a combination of tension strain gauge resistors and compression strain gauge resistors.

As depicted, the seventh half-bridge circuit 3831G can include the thirteenth strain gauge resistor (R₁₃) and the fourteenth strain gauge resistor (R₁₄). The thirteenth and fourteenth strain gauge resistors (R₁₃, R₁₄) are positioned in axial alignment with the beam center axis A_{CL}. In some embodiments, a portion of the thirteenth strain gauge resistor (R₁₃) is positioned axially between the portions of the fourteenth strain gauge resistor (R₁₄), and/or a portion of the fourteenth strain gauge resistor (R₁₄) is positioned axially between the portions of the thirteenth strain gauge resistor (R₁₃). In some embodiments, one of the thirteenth and fourteenth strain gauge resistors (R₁₃, R₁₄) can be a tension strain gauge resistor while the other is a compression strain gauge resistor. The seventh half-bridge circuit 3831G can be positioned distally relative to the fourth half-bridge circuit 3831D. In some embodiments, the seventh half-bridge circuit 3831G can be positioned proximally relative to the third half-bridge circuit 3831C.

As further depicted, the eighth half-bridge circuit 3831H can include the fifteenth strain gauge resistor (R₁₅) and the sixteenth strain gauge resistor (R₁₆). The fifteenth and sixteenth strain gauge resistors (R₁₅, R₁₆) are positioned in axial alignment with the beam center axis A_{CL}. In some embodiments, a portion of the fifteenth strain gauge resistor (R₁₅) is positioned axially between the portions of the sixteenth strain gauge resistor (R₁₆), and/or a portion of the sixteenth strain gauge resistor (R₁₆) is positioned axially between the portions of the fifteenth strain gauge resistor (R₁₅). In some embodiments, one of the fifteenth and sixteenth strain gauge resistors (R₁₅, R₁₆) is a tension strain gauge resistor while the other is a compression strain gauge resistor. The eighth half-bridge circuit 3831H can be positioned proximally relative to the third half-bridge circuit 3831C.

In some embodiments, an output of the first secondary-full-bridge circuit can be redundant to a corresponding output of the first primary-full-bridge circuit. Similarly, an output of the second secondary-full-bridge circuit can be redundant to a corresponding output of the second primary-full-bridge circuit. In other words, absent a sensor malfunction, the respective outputs of the first and second secondary-full-bridge circuits substantially equal the respective outputs of the corresponding first and second primary-full-bridge circuits.

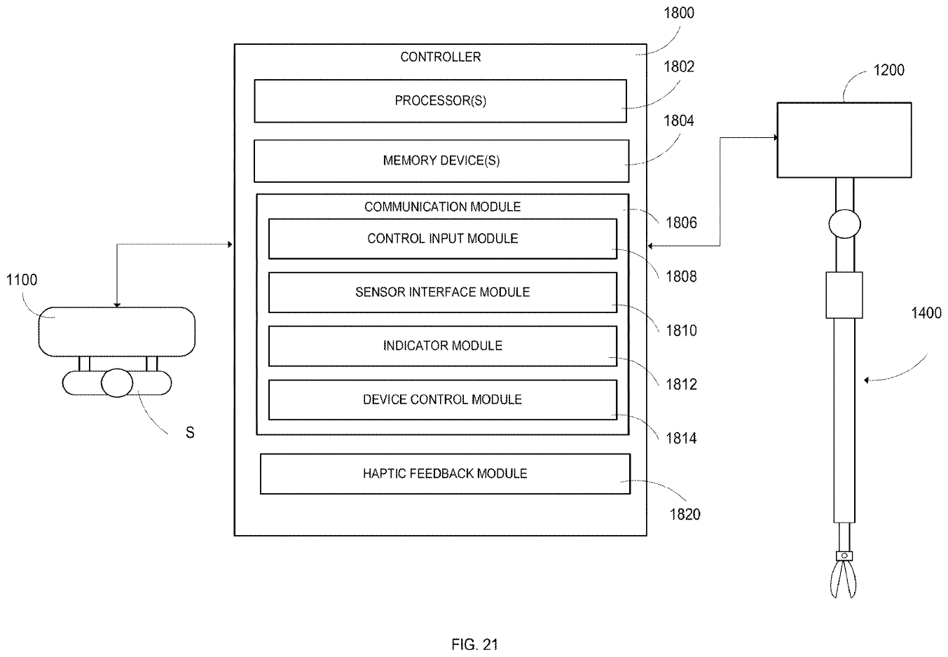

As shown particularly in FIG. 21, a schematic diagram of one embodiment of suitable components that may be included within the controller 1180 is illustrated. In some embodiments, the controller 1180 is positioned within a component of the surgical system 1000, such as the user control unit 1100 and/or the optional auxiliary equipment unit 1150. However, the controller 1180 may also include distributed computing systems wherein at least one aspect of the controller 1180 is at a location which differs from the remaining components of the surgical system 1000 for example, at least a portion of the controller 1180 may be an online controller.

As depicted, the controller 1180 includes one or more processor(s) 1182 and associated memory device(s) 1184 configured to perform a variety of computer implemented functions (e.g., performing the methods, steps, calculations and the like and storing relevant data as disclosed herein). Additionally, in some embodiments, the controller 1180 includes a communication module 1186 to facilitate communications between the controller 1180 and the various components of the surgical system 1000.

As used herein, the term “processor” refers not only to integrated circuits referred to in the art as being included in a computer, but also refers to a controller, a microcontroller, a microcomputer, a programmable logic controller (PLC), an application specific integrated circuit, and other programmable circuits. Additionally, the memory device(s) 1184 may generally comprise memory element(s) including, but not limited to, computer readable medium (e.g., random access memory (RAM)), computer readable nonvolatile medium (e.g., a flash memory), a floppy disc, a compact disc read only memory (CD ROM), a magneto optical disc (MOD), a digital versatile disc (DVD) and/or other suitable memory elements. Such memory device(s) 1184 may generally be configured to store suitable computer readable instructions that, when implemented by the processor(s) 1182, configure the controller 1180 to perform various functions.

In some embodiments, the controller 1180 includes a haptic feedback module 1196. The haptic feedback module 1196 may be configured to deliver a haptic feedback to the operator based on inputs received from a force sensor unit 1800 of the instrument 1400. In some embodiments, haptic feedback module 1196 may be an independent module of the controller 1180. However, in some embodiments the haptic feedback module 1196 may be included within the memory device(s) 1184.

The communication module 1186 may include a control input module 1188 configured to receive control inputs from the operator/surgeon S, such as via the input device 1116 of the user control unit 1100. The communication module may also include an indicator module 1192 configured to generate various indications in order to alert the operator.

The communication module 1186 may also include a sensor interface 1190 (e.g., one or more analog to digital converters) to permit signals transmitted from one or more sensors (e.g., strain sensors of the force sensor unit 1800) to be converted into signals that can be understood and processed by the processors 1182. The sensors may be communicatively coupled to the communication module 1186 using any suitable means. For example, the sensors may be coupled to the communication module 1186 via a wired connection and/or via a wireless connection, such as by using any suitable wireless communications protocol known in the art. Additionally, in some embodiments, the communication module 1186 includes a device control module 1814 configured to modify an operating state of the instrument 1400 (and/or any of the instruments described herein. Accordingly, the communication module is communicatively coupled to the manipulator unit 1200 and/or the instrument 1400. For example, the communications module 1186 may communicate to the manipulator unit 1200 and/or the instrument 1400 an excitation voltage for the strain sensor(s), a handshake and/or excitation voltage for a positional sensor (e.g., for detecting the position of the designated portion relative to the cannula), cautery controls, positional setpoints, and/or an end effector operational setpoint (e.g., gripping, cutting, and/or other similar operation performed by the end effector).

While various embodiments have been described above, it should be understood that they have been presented by way of example only, and not limitation. Where methods and/or schematics described above indicate certain events and/or flow patterns occurring in certain order, the ordering of certain events and/or operations may be modified. While the embodiments have been particularly shown and described, it will be understood that various changes in form and details may be made.

For example, any of the instruments described herein (and the components therein) are optionally parts of a surgical assembly that performs minimally invasive surgical procedures, and which can include a manipulator unit, a series of kinematic linkages, a set of cannulas, or the like. Thus, any of the instruments described herein can be used in any suitable surgical system, such as the MIRS system 1000 shown and described above. Moreover, any of the instruments shown and described herein can be used to manipulate target tissue during a surgical procedure. Such target tissue can be cancer cells, tumor cells, lesions, vascular occlusions, thrombosis, calculi, uterine fibroids, bone metastases, adenomyosis, or any other bodily tissue. The presented examples of target tissue are not an exhaustive list. Moreover, a target structure can also include an artificial substance (or non-tissue) within or associated with a body, such as for example, a stent, a portion of an artificial tube, a fastener within the body or the like.

For example, any of the components of a surgical instrument as described herein can be constructed from any material, such as medical grade stainless steel, nickel alloys, titanium alloys or the like. Further, any of the links, tool members, beams, shafts, cables, or other components described herein can be constructed from multiple pieces that are later joined together. For example, in some embodiments, a link can be constructed by joining together separately constructed components. In other embodiments however, any of the links, tool members, beams, shafts, cables, or components described herein can be monolithically constructed.

Although various embodiments have been described as having particular features and/or combinations of components, other embodiments are possible having a combination of any features and/or components from any of embodiments as discussed above. Aspects have been described in the general context of medical devices, and more specifically surgical instruments, but inventive aspects are not necessarily limited to use in medical devices.

1. A force sensing medical instrument, comprising:
a proximal mechanical structure;
an instrument shaft coupled to the proximal mechanical structure;
a force sensor unit coupled to a distal end portion of the instrument shaft;
a circuit board coupled to the proximal mechanical structure and configured to receive an output from the force sensor unit; and
a sensor cable having a middle portion and a first plurality of electrical traces communicatively coupled between the force sensor unit and the circuit board;
wherein the first plurality of electrical traces includes an electrical ground trace;
wherein the sensor cable includes an electrical shield that surrounds the middle portion of the sensor cable; and
wherein the electrical shield is communicatively coupled to the electrical ground trace.

2. The medical instrument of claim 1, wherein:
the sensor cable includes a proximal end portion, a distal end portion, a first layer, a proximal second layer, and a distal second layer separate from the proximal second layer;
the first layer extends between the distal end portion of the sensor cable and the proximal end portion of the sensor cable and includes a proximal segment and a distal segment;
the proximal second layer extends parallel to the proximal segment of the first layer;
the distal second layer extends parallel to the distal segment of the first layer;
the proximal segment of the first layer includes a proximal coupling interface, and the proximal coupling interface includes a first plurality of electrically conductive contacts;
the proximal segment of the first layer in the proximal end portion of the sensor cable is free of the first plurality of electrical traces; and
the proximal second layer is coupled to the proximal segment and contains a second plurality of electrical traces communicatively coupled to the plurality of conductive contacts.

3. The medical instrument of claim 2, wherein:
the middle portion of the sensor cable is free of the proximal second layer and the distal second layer; and
the first plurality of electrical traces extends in a side-by-side planar configuration within the first layer through the middle portion of the sensor cable.

4. The medical instrument of claim 3, wherein:
the sensor cable includes a proximal transfer portion between the proximal end portion and the middle portion of the sensor cable; and
the proximal transfer portion includes a plurality of vias configured to communicatively couple the first plurality of electrical traces in the first layer in the middle portion to the second plurality of electrical traces in the proximal second layer.

5. The medical instrument of claim 3, wherein:
the first plurality of electrical traces includes a plurality of positive traces and a plurality of negative traces in the middle portion of the sensor cable;
each positive trace of the plurality of positive traces has a first cross-sectional area;
each negative trace of the plurality of negative traces has a second cross-sectional area; and
the first cross-sectional area is smaller than the second cross-sectional area.

6. The medical instrument of claim 5, wherein:
a first maximal resistance limit determines a minimal first cross-sectional area of the first cross-sectional area of the plurality of positive traces; and
a second maximal resistance limit determines a minimal second cross-sectional area of the second cross-sectional area of the plurality of negative traces.

7. The medical instrument of claim 5, wherein:
a maximal sensor cable width defines a maximal combined cross-sectional area of each of the plurality of positive traces and the plurality of negative traces in the middle portion of the sensor cable; and
the maximal sensor cable width is defined at least in part by a passage clearance of the instrument shaft.

8. The medical instrument of claim 2, wherein:
the sensor cable includes a first electrically insulative layer, an electrically insulative base, and a second electrically insulative layer;
the first electrically insulative layer is on the distal segment of the first layer and the middle portion of the sensor cable;
the first electrically insulative layer is absent from the proximal segment of the first layer;
the electrically insulative base extends between the distal end portion and the proximal end portion of the sensor cable; and
the second electrically insulative layer is on the distal second layer, the middle portion of the sensor cable, and the proximal second layer.

9. The medical instrument of claim 2, wherein:
the sensor cable includes a longitudinal axis extending between the proximal end portion and the distal end portion; and
the first plurality of conductive contacts is arranged along a contact axis that is parallel to the longitudinal axis of the sensor cable.

10. The medical instrument of claim 2, wherein:
the proximal coupling interface is an anisotropic conductive film coupling.

11. The medical instrument of claim 2, wherein:
the sensor cable includes a distal transfer portion between the middle portion and the distal end portion of the sensor cable; and
the distal transfer portion includes a plurality of vias configured to communicatively couple a first portion of the first plurality of electrical traces in the middle portion to a third plurality of electrical traces in the distal second layer.

12. The medical instrument of claim 11, wherein:
the distal segment of the first layer in the distal end portion of the sensor cable includes a distal coupling interface, the distal coupling interface having a second plurality of conductive contacts coupled to the force sensor unit;
a second portion of the first plurality of electrical traces are communicatively coupled to the distal coupling interface in the first layer;
the third plurality of electrical traces in the distal second layer is coupled to the distal coupling interface; and
a linear arrangement of the second plurality of conductive contacts establishes an initial configuration of the third plurality of electrical traces and the second portion of the first plurality of electrical traces.

13. The medical instrument of claim 12, wherein:
the third plurality of electrical traces and the second portion of the first plurality of electrical traces are rearranged within the distal transfer portion to establish all electrical traces of the first plurality of electrical traces in a side-by-side, planar configuration within the first layer through the middle portion of the sensor cable.

14. The medical instrument of claim 13, wherein:
the middle portion of the sensor cable includes a first lateral side region and a second lateral side region separated by the electrical ground trace;
the first plurality of electrical traces includes a plurality of positive traces and a plurality of negative traces in the middle portion of the sensor cable; and
the side-by-side, planar configuration includes the plurality of positive traces positioned within the first lateral side region and the plurality of negative traces positioned within the second lateral side region.

15. The medical instrument of claim 13, wherein:
the first plurality of electrical traces includes one or more positive traces and one or more negative traces in the middle portion of the sensor cable; and
the side-by-side, planar configuration includes the one or more positive traces being arranged in a positive-negative pairing with the one or more negative traces.

16. The medical instrument of claim 12, wherein:
the medical instrument further comprises a beam, and the beam includes a first face and a second face;
the force sensor unit includes a strain sensor on the first face of the beam;
the distal coupling interface is coupled to the strain sensor on the first face of the beam;
the distal end portion of the sensor cable is coupled to the second face of the beam adjacent the first face of the beam;
the distal coupling interface is formed with a pre-fold relative to a remainder of the distal end portion to align the distal coupling interface with the first face of the beam; and
a magnitude of the pre-fold corresponds to an angle between the first face of the beam and the second face of the beam.

17. The medical instrument of claim 2, wherein:
the sensor cable includes a balancing portion that extends distally from the distal end portion of the sensor cable;
the balancing portion has a stiffness that corresponds to a stiffness of the distal end portion of the sensor cable; and
the balancing portion has an absence of electrical traces.

18. The medical instrument of claim 2, wherein:
the medical instrument further comprises a beam, and the beam includes a first face, a second face, and a third face;
the force sensor unit includes a strain sensor on the first face of the beam;
the distal end portion of the sensor cable is coupled to the second face of the beam adjacent the first face of the beam;
the sensor cable includes a stiffness-balancing tab coupled to the third face of the beam;
the stiffness-balancing tab has a stiffness that corresponds to one of a stiffness of the strain sensor or a stiffness of the distal end portion of the sensor cable; and
the stiffness-balancing tab has an absence of electrical traces.

19. The medical instrument of claim 18, wherein:
the strain sensor includes eight bridge circuits arranged as four bridge-circuit combinations; and
each bridge circuit of the eight bridge circuits includes two strain gauges.

20-21. (canceled)

22. A surgical system, comprising:
the medical instrument of claim 1, the medical instrument including an end effector, a wrist assembly, and a manipulator unit, wherein:
the end effector is coupled to the force sensor unit via the wrist assembly,
the manipulator unit is configured to manipulate the medical instrument, and
the medical instrument is removably coupled to the manipulator unit.
